(19) United States
(12) Patent Application Publication
(10) Pub. No.: US 2023/0295227 A1
(43) Pub. Date: Sep. 21, 2023

(54) COMPOUNDS FOR USE IN DIAGNOSIS AND/OR MONITORING OF FIBROSIS

(71) Applicants: ANTAROS TRACER AB, Mölndal (SE); Antaros Tracer AB, Mölndal (SE)

(72) Inventors: Olof ERIKSSON, Uppsala (SE); Olov KORSGREN, Uppsala (SE); Christer WESTERLUND, Mölndal (SE); Michael WAGNER, Mölndal (SE)

(21) Appl. No.: 18/010,904

(22) PCT Filed: Jun. 28, 2021
(86) PCT No.: PCT/EP2021/067653

§ 371 Date: Dec. 16, 2022

(30) Foreign Application Priority Data
Jun. 29, 2020 (SE) 2050786-9

(51) Int. Cl. C07K 7/04; A61K 51/08; C07D 257/02; C07D 255/02; A61B 6/03

(52) U.S. Cl. CPC C07K 7/04; A61K 51/088; C07D 257/02; C07D 255/02; A61B 6/037

(57) ABSTRACT

There is provided a composition comprising: (i) a compound of Formula I: (I), or a pharmaceutically acceptable salt thereof, and (ii) a nuclide M, or a pharmaceutically acceptable salt of the compound of Formula I and/or the nuclide M, wherein C is a chelator selected from the group consisting of 1,4,7,10-tetraazacyclododecane-1,4,7,10-tetraacetic acid (DOTA), 1,4,7-triazacyclononane-1,4,7-triacetic acid (NOTA), 1,4,8,11-tetraazacycl otetradecane-1,4,8,11-tetraacetic acid (TETA), diethylenetriaminepentaacetic acid (DTPA), desferrioxamine B (DFO), 1,4,7-Triazacyclononane-1-glutaric acid-4,7-acetic acid (NOTAGA), 2-[4,7,10-Tris(carboxymethyl)-1,4,7,10-tetraza-1-cyclododecyl]glutaric acid (DOTAGA and a derivative of any one of the foregoing chelators, L (Formula L) is a linker: (L), wherein m is an integer within the range of from 1 to 20, and X is NH or C(O) and forms an amide bond, i.e. C(O)NH, with a C(O) or NH moiety of the chelator, p is 0 or 1, Q is a peptide of SEQ ID NO: 1, a peptide analogue of SEQ ID NO: 1 having at least 88.8% identity to SEQ ID NO. 1, and/or a peptide of SEQ ID NO: 1, a peptide analogue of SEQ ID NO: 1 having at least 88.8% identity to SEQ ID NO: 1 and in which the C-terminal COOH can be replaced by CONH₂, and M is selected from the group consisting of ⁶⁸Ga, ¹⁸F, ⁶⁴Cu, ⁴⁴Sc, ⁸⁹Zr, ¹¹¹In, ⁶⁷Ga, ^{99m}Tc, Mn, Gd, ¹⁷⁷Lu.and ^{86/90}Y. The composition may be used in in diagnosing and/or monitoring of fibrosis.

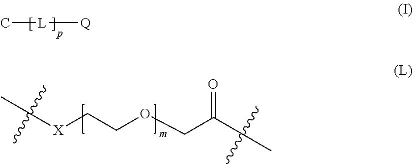

### TECHNICAL FIELD

The present disclosure concerns novel compounds comprising a non-cyclic peptide, a linker, a chelator and a nuclide such as a radionuclide. The compounds may be used as tracers such as radioactive tracers for use in the diagnosis and/or monitoring of fibrosis such as fibrosis occurring in the liver, kidney, heart, brain, pancreas, and lungs of a patient. The disclosure further relates to a method for preparing the compounds, a compound that may be used as an intermediate in the aforementioned method as well as a method for diagnosing and/monitoring of fibrosis in a patient.

### BACKGROUND

Fibrosis is the formation of connective tissues that might occur in normal physiology as a response to injury, which is known as scarring. However, excess formation and deposition of connective tissue, which constitutes the pathological formation of fibrosis, is an important feature in many different tissues in disease, e.g., liver, kidney, heart, brain, pancreas, and lungs. The pathological formation of fibrosis is due to an increase in the production and deposition of collagens, especially collagen type I, which results in loss of tissue elasticity and progressive loss of organ function. It has been found that fibrosis is involved in a large number of prevalent and severe diseases involving organs such as the liver, kidney, heart, brain, pancreas and lungs.

Current treatments against fibrotic disease, i.e., fibrosis, mainly target the inflammatory cascade, but efforts to develop novel treatments have proven very challenging. The treatment objective is to slow down the fibrotic process. To date, there are unfortunately no drugs available that can reverse fibrosis. In addition to the challenge of developing drugs targeting the inflammatory system, fibrotic disease often lacks reliable biomarkers. Several pre-clinical disease models have been developed, but in many cases, they suffer in ‘translatability’ from mice to humans. Diagnosis of fibrotic disease may be determined from a biopsy sample when this is feasible. But methods to measure changes precisely and repeatedly in the fibrotic process as required in drug development are largely lacking. For fibrotic liver disease, Magnetic Resonance Elastography (MRE) is used as a non-invasive biomarker of liver stiffness, but for most fibrotic disease such non-invasive methods are not yet available. Of course, non-invasive methods are more desirable than invasive methods, such as biopsies, since non-invasive methods are more convenient, can be performed repeatedly, and are associated with a lower risk of harming the patient. Therefore, further non-invasive diagnostic methods for detection of fibrosis have been proposed.

Nuclear Medicine and Biology, 41 (2014) 728-736 discloses synthesis and preclinical evaluation of ⁶⁸Ga-labeled collagelin analogs for imaging and quantification of fibrosis by positron emission tomography (PET). The analogs were prepared and intended for binding to collagen overexpressed in fibrotic tissues, since collagen is a biomarker that can be targeted in molecular imaging of fibrosis providing direct identification of the fibrotic tissue. It is disclosed that the tracers displayed a pronounced washout pattern from most of the organs except for kidneys and bladder.

Sci. Trans. Med. 9, 2017, 1-11 discloses a type I collagen-targeted PET probe for pulmonary fibrosis detection and staging in preclinical models. The probe used was ⁶⁸Ga—CBP8, which was found to have a specificity for type I collagen. It is stated that ⁶⁸Ga—CBP8 provided significantly enhanced PET signal in the lungs of fibrotic mice compared with control mice, and that nonspecific uptake in the surrounding tissues was similar and low in both fibrotic and control mice but with high off-target accumulation in the kidney.

WO 2018/053276 discloses polymer conjugates having utility in the treatment of a subject suffering from soft tissue conditions. The polymer conjugates comprise sulfated glycosaminoglycan chains which may be substituted with a collagen-binding agent such as a peptide with the sequence LRELHLNNN (IUPAC-IUB nomenclature).

Thus, collagens, especially collagen type 1, is known as a biomarker for fibrosis. Further, for all organs but kidney the cyclic peptides of the above-mentioned radioactive tracers have been found to have affinity for collagen while exhibiting a low background binding.

Importantly, to allow for accurate imaging of the fibrosis, the tracer such as the radioactive tracer should have a low non-specific binding to normal tissue, fast blood clearance and washout from healthy organs. Thus, there should be low or no binding to tissues lacking deposits of collagen such as collagen type 1. In other words, the biodistribution of the radiotracer should be selective so that binding mainly takes place to organs involving fibrotic tissue.

Radioactive tracers may exhibit retention in tissues for many different reasons. Retention of a collagen targeting radioactive tracer may be retained in tissues by e.g., non-specific binding to cellular components, or by specific unintended targeting of molecular entities such as receptors. Radiolabeled peptides may additionally exhibit reabsorption in the renal tubules during urinary excretion, with subsequent intracellular trapping of the radionuclide in the kidney cortex. Regardless of the cause of such tissue retention, it precludes the measurement and diagnosis of the existence and/or progression of fibrotic lesions in said tissue.

Further, in order to detect the presence of fibrosis it is important that the radioactive tracer is able to thoroughly penetrate the organ to ensure that the entire organ is investigated for fibrosis. This may be more difficult in solid organs such as liver, kidney, heart, brain, pancreas, and lungs compared to non-solid organs.

There is a need for a tracer such as a radiotracer for fibrosis with a suitable biodistribution in all or most organs such as suitable biodistribution with respect to kidney. Further, there is a need for a tracer for fibrosis which is able to penetrate the entire organ being investigated for fibrosis.

It is an object of the present disclosure to alleviate at least one or more of the problems discussed above. Further, it is an object of the present disclosure to provide advantages and/or aspects not provided by hitherto known techniques.

### SUMMARY

The above objects may be achieved with a composition in accordance with claims 1 and 2 or a compound in accordance with claim 19, and by using a method in accordance with claim 25. Further embodiments are set out in the dependent claims, the description and in the drawings.

The present disclosure provides a composition comprising:

(i) a compound of Formula I:

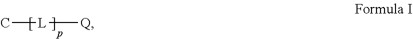

or a pharmaceutically acceptable salt thereof,

and

(ii) a nuclide M, or a pharmaceutically acceptable salt thereof,

wherein

C is a chelator selected from the group consisting of:

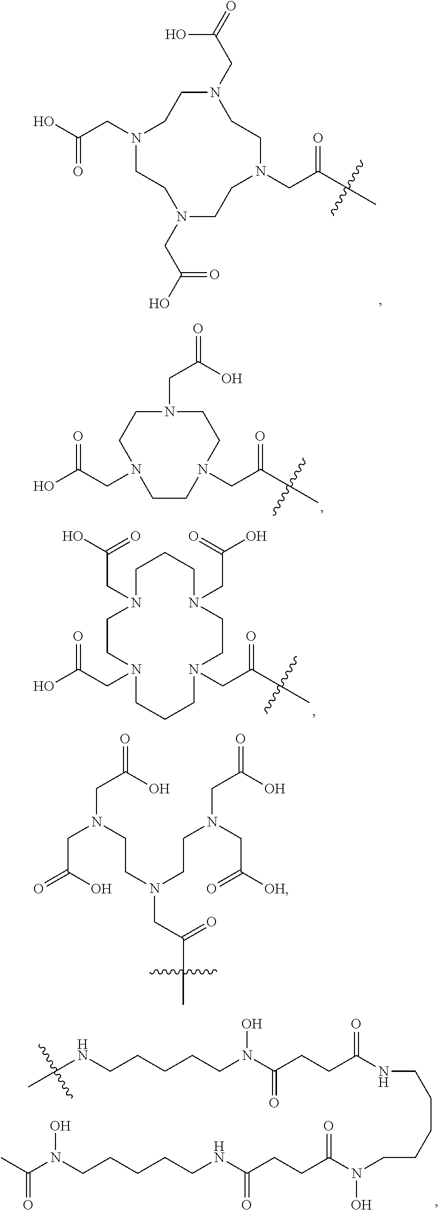

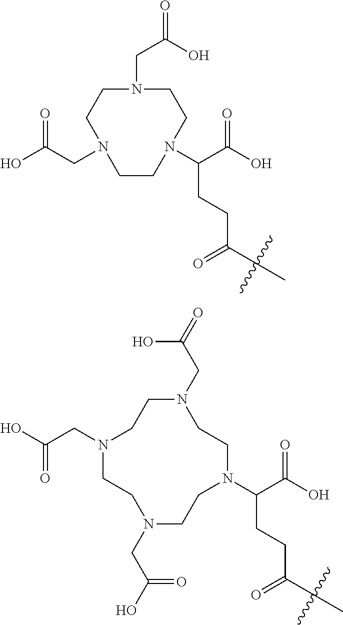 [SMILES: *C(=O)CCC(C(=O)O)N1CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

and a derivative of any one of the foregoing chelators,

L is a linker:

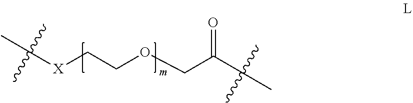

wherein

m is an integer within the range of from 1 to 20, and

X is NH or C(O) and forms an amide bond, i.e. C(O)NH, with a C(O) or NH moiety of the chelator,

p is 0 or 1,

Q is a peptide of

SEQ ID NO: 1, a peptide analogue of SEQ ID NO: 1 having at least 88.8% identity to SEQ ID NO: 1, and/or

a peptide of SEQ ID NO: 1, a peptide analogue of SEQ ID NO: 1 having at least 88.8% identity to SEQ ID NO: 1 and in which the C-terminal COOH can be replaced with CONH₂,

and

M is selected from the group consisting of ⁶⁸Ga, ¹⁸F, ⁶⁴Cu, ⁴⁴Sc, ⁸⁹Zr, ¹¹¹In, ⁶⁷Ga, ^{99m}Tc, Mn Gd, ¹⁷⁷Lu.and ^{86/90}Y.

The present disclosure also provides a compound of Formula I as described herein, or a pharmaceutically acceptable salt thereof.

Further, the present disclosure provides a compound of Formula II:

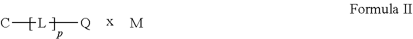

or a pharmaceutically acceptable salt thereof,

said compound of Formula II being a combination of

(i) the compound of Formula I as defined in claim 1 and

(ii) the nuclide M as defined in claim 1,

wherein (i) and (ii) are provided in a ratio (i)/(ii) equal to one.

There is also provided

a composition as described herein,

or

a compound of Formula II as described herein, or a pharmaceutically acceptable salt

thereof,

for use in diagnosing and/or monitoring of fibrosis.

There is also provided

a composition as described herein,

or

a compound of Formula II as described herein, or a pharmaceutically acceptable salt thereof,

for the manufacture of a preparation for the diagnosis and/or monitoring of fibrosis.

There is also provided a use of

a composition as described herein,

or

a compound of Formula II as described herein, or a pharmaceutically acceptable salt thereof,

for diagnosing and/or monitoring of fibrosis such as diagnosing and/or monitoring fibrosis in a patient suffering from, suspected to be suffering from and/or being treated for, fibrosis.

Further, there is provided a method for the diagnosis and/or monitoring of fibrosis, said method comprising the steps of:
a) administering an imaging agent selected from one or more of the following:
   a composition as described herein,
   a compound of Formula II as described herein,
   a pharmaceutically acceptable salt of a compound of Formula II as described herein,
   to a patient suffering from, suspected to be suffering from and/or being treated for fibrosis;
b) subjecting the patient to a medical imaging technique, such as Positron Emission Tomography (PET), Single-Photon Emission Computed Tomography (SPECT) or Magnetic Resonance Imaging (MRI) imaging, and recording signals from the imaging agent administered in step a);
c) determining and/or monitoring if the patient suffers from fibrosis, and
d) optionally determining the extent of the fibrosis.

### BRIEF DESCRIPTION OF THE DRAWINGS

FIG. 1 shows the chemical structure of DOTA, i.e. 1,4,7,10-tetraazacyclododecane-1,4,7,10-tetraacetic acid.

FIG. 2 shows the chemical structure of NOTA, i.e. 1,4,7-triazacyclononane-1,4,7-triacetic acid.

FIG. 3 shows the chemical structure of TETA, i.e. 1,4,8,11-tetraazacyclotetradecane-1,4,8,11-tetraacetic acid.

FIG. 4 shows the chemical structure of DTPA, i.e. diethylenetriaminepentaacetic acid.

FIG. 5 shows the chemical structure of DFO, i.e. desferrioxamine B.

FIG. 6 shows the chemical structure of NOTAGA.

FIG. 7 shows the chemical structure of DOTAGA.

FIG. 8 shows the chemical structure of compound 1.

FIG. 9 shows the chemical structure of compound 14.

FIG. 10a shows the total and non-specific binding of [⁶⁸Ga]Ga-DOTA-NH—(CH₂CH₂O)₂—CH₂—C(O)-LRELHLNNN—OH to hepatic tissue with induced fibrosis compared to non-fibrotic liver.

FIG. 10b shows the magnitude of binding of [⁶⁸Ga]Ga-DOTA-NH—(CH₂CH₂O)₂—CH₂—C(O)-LRELHLNNN—OH to hepatic tissue and the correlation to the degree of fibrosis.

FIG. 11 shows the biodistribution of [⁶⁸Ga]Ga-DOTA-NH—(CH₂CH₂O)₂—CH₂—C(O)-LRELHLNNN—OH in rats.

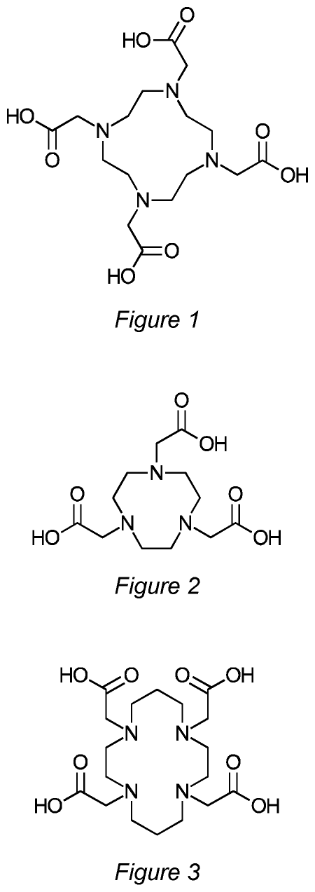

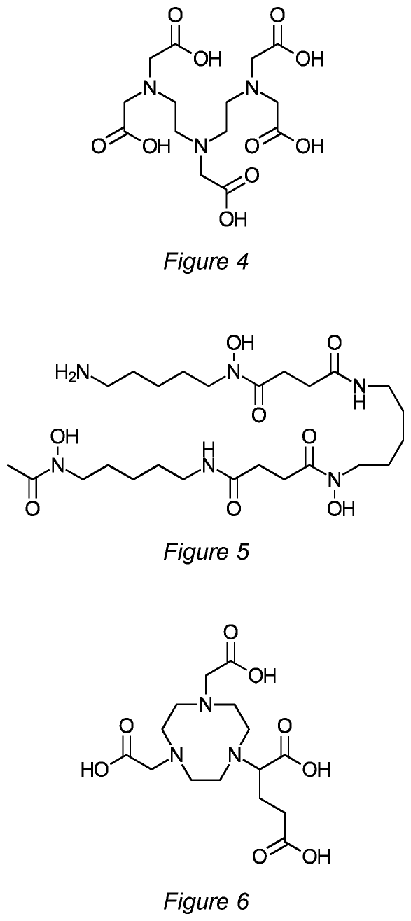

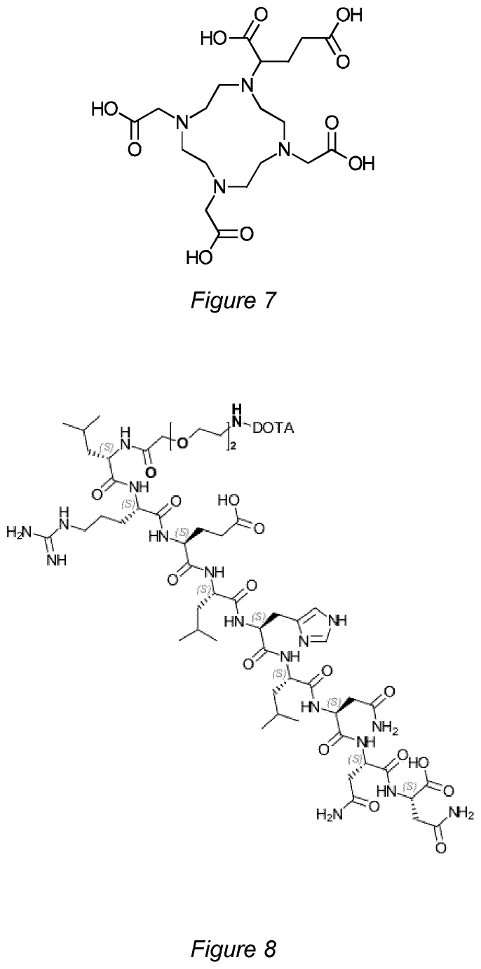

### DESCRIPTION

The present disclosure provides a composition comprising or consisting of:

(i) a compound of Formula I:

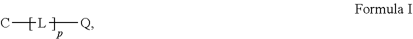

or a pharmaceutically acceptable salt thereof,

and

(ii) a nuclide M, or a pharmaceutically acceptable salt thereof,

wherein

C is a chelator selected from the group consisting of:

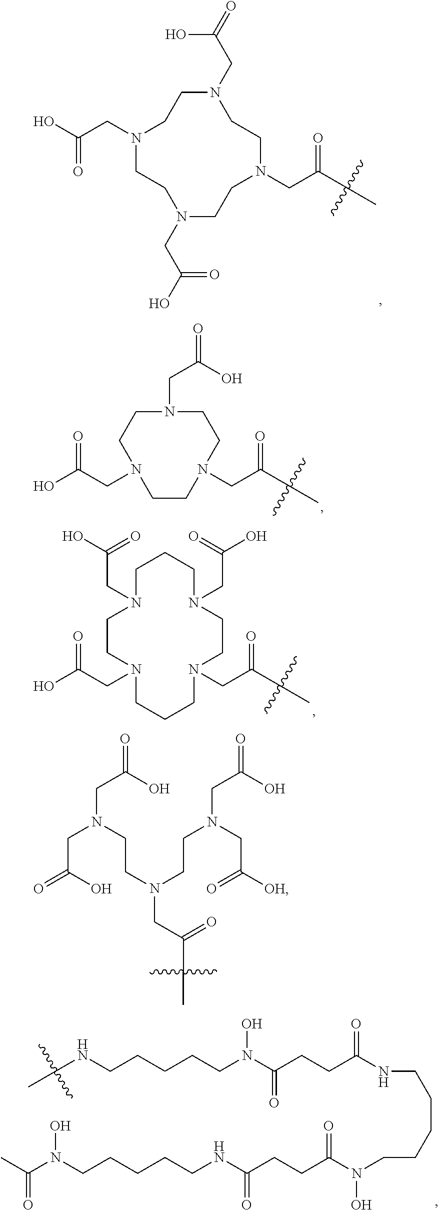

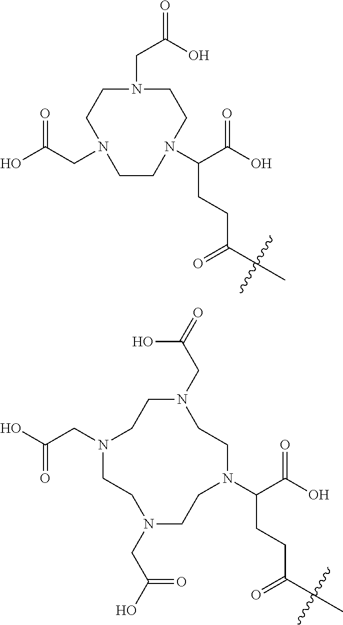 [SMILES: *C(=O)CCC(C(=O)O)N1CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

and a derivative of any one of the foregoing chelators,

L is a linker:

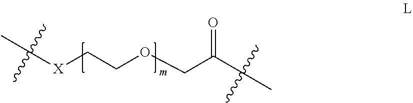

wherein

m is an integer within the range of from 1 to 20, and

X is NH or C(O) and forms an amide bond, i.e. C(O)NH, with a C(O) or NH moiety of the chelator,

p is 0 or 1,

Q is a peptide of

SEQ ID NO: 1, a peptide analogue of SEQ ID NO: 1 having at least 88.8% identity to SEQ ID NO: 1, and/or

a peptide of SEQ ID NO: 1, a peptide analogue of SEQ ID NO: 1 having at least 88.8% identity to SEQ ID NO: 1 and in which the C-terminal COOH can be replaced with CONH₂,

and

M is selected from the group consisting of ⁶⁸Ga, ¹⁸F, ⁶⁴Cu, ⁴⁴Sc, ⁸⁹Zr, ¹¹¹In, ⁶⁷Ga, ^{99m}Tc, Mn Gd, ¹⁷⁷Lu and ^{86/90}Y.

The composition described herein may comprise a compound of Formula II:

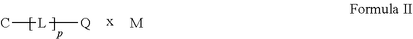

or a pharmaceutically acceptable salt thereof,

said compound being a combination of

(i) the compound of Formula I as described herein, and

(ii) the nuclide M as described herein.

The ratio between the compound of Formula I and the nuclide M in the compound of Formula II, i.e. the ratio (i)/(ii), may be equal to one. Thus, there is provided a composition as described herein in which the ratio between the compound of Formula I and the nuclide M in the compound of Formula II, i.e. the ratio (i)/(ii), is equal to one. However, it may not always be possible to control the stoichiometry and therefore the compound of Formula I and the nuclide M may be combined in unequal amounts, such as unequal molar amounts, resulting in a composition comprising the aforementioned compound of Formula II, in which the ratio between the compound of Formula I and the nuclide is one, together with an additional amount of the compound of Formula I and/or nuclide M.

While not wishing to be bound by any specific theory, it is believed that the compounds described herein such as the compound of Formula I or the compound of Formula II act by binding to collagen I. As a result, the aforementioned compounds or the composition comprising the aforementioned compounds may be used as an imaging agent for fibrosis such as fibrosis described herein.

The compounds described herein may comprise or consist of a chelator selected from the group consisting of: 1,4,7,10-tetraazacyclododecane-1,4,7,10-tetraacetic acid (DOTA), 1,4,7-triazacyclononane-1,4,7-triacetic acid (NOTA), 1,4,8,11-tetraazacyclotetradecane-1,4,8,11-tetraacetic acid (TETA), diethylenetriaminepentaacetic acid (DTPA), desferrioxamine B (DFO), 1,4,7-Triazacyclononane-1-glutaric acid-4,7-acetic acid (NOTAGA), 2-[4,7,10-Tris(carboxymethyl)-1,4,7,10-tetraza-1-cyclododecyl]glutaric acid (DOTAGA) and a derivative thereof. The derivative may include exchange of one or more carboxylic acids into an amide or ester. In a further example, DOTAGA may be used instead of DOTA. When the chelator of the compounds described herein is based on DOTA, NOTA, TETA, DTPA, NOTAGA or DOTAGA a hydroxyl group of one of the carboxylic acids is exchanged for NH through which binding to the linker takes place. When the chelator of the compounds described herein is DFO it binds via its terminal amino group to the linker's carbonyl group. As used herein, a carbonyl group may be denoted CO or C(O).

It will be appreciated that the value of the integer m of the compounds disclosed herein may be an integer within the above-mentioned range, i.e. from 1 to 20. In an example, m is 1, 2 or 3.

As described herein, the linker L comprises X which may be NH or C(O) forming an amide bond, i.e. C(O)NH, with a C(O) or NH moiety of the chelator. Thus, when X is NH it binds to a C(O) moiety, i.e. a carbonyl group, of the chelator. Further, when X is C(O) it binds to a NH moiety of the chelator.

Further, as described herein the linker L is:

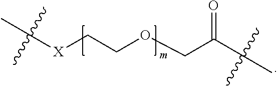

Thus, the linker L may be drafted as —X—(CH₂CH₂O)ₘ—CH₂—C(O)—. It follows that the compound of Formula I may be drafted as Chelator-[X—(CH₂CH₂O)ₘ—CH₂—C(O)]ₚ-Q. For instance, when the chelator C is DOTA, X is NH, m is 2, p is 1 and Q is LRELHLNNN the compound of Formula I may be drafted DOTA-NH—(CH₂CH₂O)₂—CH₂—C(O)-LRELHLNNN—OH.

The peptide Q of the compounds described herein may comprise or consist of a peptide (i.e. an amino acid sequence) according to SEQ ID NO: 1 (LRELHLNNN) or an analogue of SEQ ID NO: 1 in which the C-terminal COOH is replaced with CONH₂. When the C-terminal COOH is replaced by CONH₂, the sequence is written e.g. -LRELHLNNN—NH₂ Alternatively, the peptide Q of the compounds described herein may comprise or consist of a peptide having at least 88.8% identity to SEQ ID NO: 1 or a sequence having at least 88.8% identity to an analogue of SEQ ID NO: 1 in which the C-terminal COOH is replaced with CONH₂. In the context of the present document, by a peptide having an amino acid sequence with at least 88.8% identity to an amino acid sequence of SEQ ID NO: 1 is intended a peptide that is identical to SEQ ID NO: 1, except that the amino acid sequence of SEQ ID NO: 1 may include one amino acid change. The one amino acid change may involve a natural amino acid, i.e. an L amino acid, or a D amino acid. In other words, to obtain a peptide having an amino acid sequence at least 88.8% identical to SEQ ID NO: 1, one amino acid in SEQ ID NO: 1 may be deleted, extended, or substituted with another amino acid, or one amino acid is inserted into SEQ ID NO: 1. The amino acid used for the substitution, extension or insertion may be a natural amino acid or a D amino acid. These amino acid changes of the SEQ ID NO: 1 may occur either at the amino or carboxy terminal position or anywhere between those terminal positions interspersed individually among amino acids in the SEQ ID NO: 1.

The letters in the peptide LRELHLNNN are the usual amino acid letters in which each amino acid is in L configuration, i.e. natural amino acids. Thus, LRELHLNNN intends a sequence Leu-Arg-Glu-Leu-His-Leu-Asn-Asn-Asn in which all amino acids are natural amino acids. In this document, Leu stands for leucine, Arg stands for arginine, Glu stands for glutamic acid, His stands for histidine and Asn stands for asparagine. The peptide Q is a non-cyclic peptide.

The percent identity between two amino acid or polynucleotide sequences is determined by dividing the number of matches by the length of the sequence set forth in an identified sequence followed by multiplying the resulting value by 100. The terms “% identity”, “% identical”, and the like, as used throughout this document, may for example be calculated as follows: The query sequence is aligned to the target sequence using the CLUSTAL W algorithm (Thompson et al., (1994) Nucleic Acids Research, 22: 4673-4680). A comparison is made over the window corresponding to the shortest of the aligned sequences. The shortest of the aligned sequences may in some instances be the target sequence. In other instances, the query sequence may constitute the shortest of the aligned sequences. The amino acid residues at each position are compared and the percentage of positions in the query sequence that have identical correspondences in the target sequence is reported as % identity.

The amino acids of the peptide Q may be either in the L configuration, i.e. natural amino acids (denoted in uppercase letters), or in the D configuration. Amino acids having a D configuration are denoted with lowercase letters. Further examples of peptide Q of the compound of Formula I described herein are listed in Table I below.

The amino acids of Q may be described with one letter code as known in the art so that the Q may also be described as LRELHLNNN. It will be understood that in the compounds described herein are straight (i.e. non-cyclic) peptides, which are drafted so that the N-terminal is at the left-hand side and the C-terminal at the right hand side.

The linker L may bind to any amino group on one of the amino acids of Q. Either one of the hydrogens of the N-terminal amino group may be replaced with a bond to the linker L, or, alternatively, the linker L may form a bond by replacing one of the hydrogens of a side chain amino group, e.g. in a Lysine situated in any position in Q,

There is also provided a composition as described herein, wherein the compound of Formula I is selected from the group consisting of a compound of Formula Ia, Formula Ib, Formula Ic, Formula Id, Formula Ie, Formula If or Formula Ig

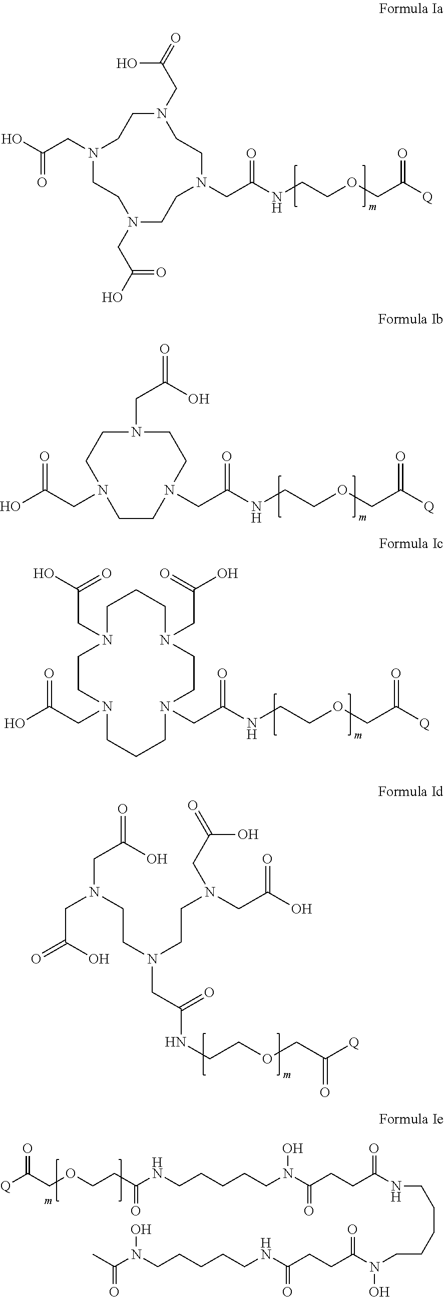

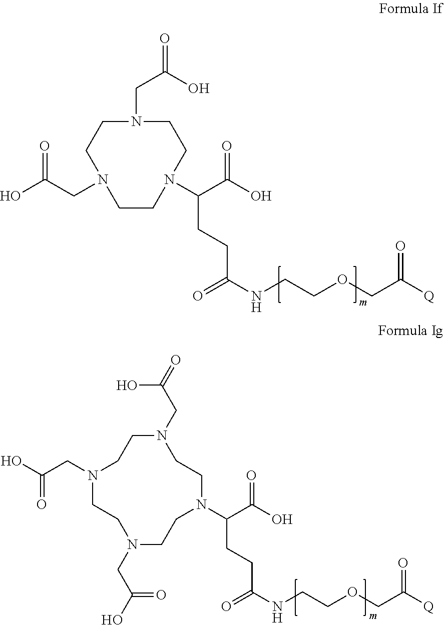

or a derivative of any one of the foregoing compounds,

or a pharmaceutically acceptable salt of any one of the foregoing compounds or a derivative of any one of the foregoing compounds.

The present disclosure also provides a compound of Formula I as described herein. Thus, there is provided a compound of Formula I:

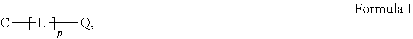

or a pharmaceutically acceptable salt thereof,

wherein C, L, p, and Q are as described herein.

For example, when p is zero the structure of the compound of Formula I is C-Q, i.e. no linker is present When p is one the structure of the compound of Formula I is C-L-Q.

Compounds of Formula I may have the following structures:

DOTA-NH—(CH₂CH₂O)₂—CH₂—C(O)-L-R-E-L-H-L-N—N—N—OH (see FIG. 8) Compound 1

DOTA-NH—(CH₂CH₂O)₂—CH₂—C(O)-L-R-E-L-H-L-N-A-N—OH Compound 2

DOTA-NH—(CH₂CH₂O)₂—CH₂—C(O)-L-R-E-L-H-L-N—N—N—NH₂ Compound 3

DOTA-NH—(CH₂CH₂O)₂—CH₂—C(O)-L-R-E-L-H-L-N—N-n-OH Compound 4

DOTA-NH—(CH₂CH₂O)₂—CH₂—C(O)-L-R-E-L-H-L-N-A-N—NH₂ Compound 5

DOTA-NH—(CH₂CH₂O)₂—CH₂—C(O)-L-R-E-L-H—V-N—N—N—OH Compound 6

DOTA-NH—(CH₂CH₂O)₂—CH₂—C(O)-L-R-E-1-H-L-N—N—N—OH Compound 7

DOTA-NH—(CH₂CH₂O)₂—CH₂—C(O)-L-R-E-L-H-L-N—N—OH Compound 8

DOTA-NH—(CH₂CH₂O)₂—CH₂—C(O)—H-L-R-E-L-H-L-N—N—N—OH Compound 9

DOTA-NH—(CH₂CH₂O)₂—CH₂—C(O)-L-R-E-L-H-L-N—N—N—K—OH Compound 10

DOTA-NH—(CH₂CH₂O)₃—CH₂—C(O)-L-R-E-L-H-L-N—N—N—OH Compound 11

DOTA-L-R-E-L-H-L-N—N—N—OH Compound 12

H₂N-L-R-E-L-H-L-N—N—N—K(DOTA-NH—(CH₂CH₂O)₂—CH₂—C(O))—NH₂ Compound 13

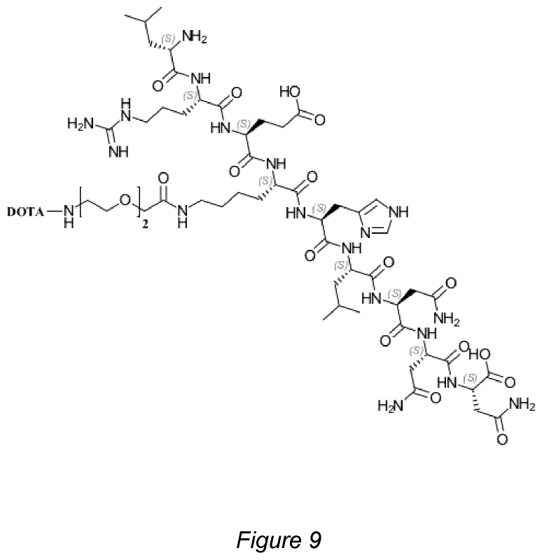

H₂N-L-R-E-K(DOTA-NH—(CH₂CH₂O)₂—CH₂—C(O))—H-L-N—N—N—OH (see FIG. 9) Compound 14

20 H₂N-L-R-E-L-H—K(DOTA-NH—(CH₂CH₂O)₂—CH₂—C(O))—N—N—N—OH Compound 15

NOTA-NH—(CH₂CH₂O)₂—CH₂—C(O)-L-R-E-L-H-L-N—N—N—OH Compound 16

The chelators, linkers, peptides and peptide C-terminal of the compounds 1-16 are summarized in Table I below:

TABLE I

| Compound | | | | |
|---|---|---|---|---|
| no. | Chelator | Linker | Peptide | C-terminal |
| 1 | DOTA- | —NH—(CH₂CH₂O)₂—CH₂—C(O)— | -LRELHLNNN- | —OH |
| 2 | DOTA- | —NH—(CH₂CH₂O)₂—CH₂—C(O)— | - LRELHLNAN- | —OH |
| 3 | DOTA- | —NH—(CH₂CH₂O)₂—CH₂—C(O)— | - LRELHLNNN- | —NH₂ |
| 4 | DOTA | —NH—(CH₂CH₂O)₂—CH₂—C(O)— | -LRELHLNNn - | —OH |
| 5 | DOTA- | —NH—(CH₂CH₂O)₂—CH₂—C(O)— | -LRELHLNAN- | —NH₂ |
| 6 | DOTA- | —NH—(CH₂CH₂O)₂—CH₂—C(O)— | LRELHVNNN | —OH |
| 7 | DOTA- | —NH—(CH₂CH₂O)₂—CH₂—C(O)— | -LREIHLNNN- | —OH |
| 8 | DOTA- | —NH—(CH₂CH₂O)₂—CH₂—C(O)— | -LRELHLNN-- | —OH |
| 9 | DOTA- | —NH—(CH₂CH₂O)₂—CH₂—C(O)— | -HLRELHLNNN- | —OH |
| 10 | DOTA- | —NH—(CH₂CH₂O)₂—CH₂—C(O)— | -LRELHLNNNK- | —OH |
| 11 | DOTA- | —NH—(CH₂CH₂O)₃—CH₂—C(O)— | -LRELHLNNN- | —OH |
| 12 | DOTA- | — | -LRELHLNNN- | —OH |
| 13 | DOTA- | —NH—(CH₂CH₂O)₂—CH₂—C(O)*— | LRELHLNNNK*- | —NH₂ |
| 14 | DOTA- | —NH—(CH₂CH₂O)₂—CH₂—C(O)*— | LREK*HLNNN- | —OH |
| 15 | DOTA- | —NH—(CH₂CH₂O)₂—CH₂—C(O)*— | LRELHK*NNN- | —OH |
| 16 | NOTA- | —NH—(CH₂CH₂O)₂—CH₂—C(O)— | -LRELHLNNN- | —OH |

For the compounds 13, 14 and 15 the linker is not attached to the N-terminal of the peptide but instead to the amino side chain of a lysine situated in different positions in the peptide. The linked parts are notated with an * both in the linker and in the lysine that are attached to each other. Parts marked in bold denotes changes in the compound of Formula I (i.e. changes in the chelator (C), the linker (L) or the peptide sequence (Q) of SEQ ID NO: 1) compared to Compound no. 1 in Table 1.

There is also provided a compound of Formula II as described herein. Thus, there is provided a compound of Formula II:

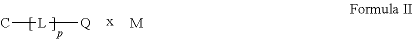

or a pharmaceutically acceptable salt thereof,

wherein C, L, p, Q and M are as described herein,

said compound of Formula II being a combination of

(i) the compound of Formula I as described herein, and

(ii) the nuclide M as described herein

In the compound of Formula II, the compound of Formula I and the nuclide M may be provided in a ratio equal to one, i.e. 1/1. Further, the compound of Formula II may be provided in admixture with an additional amount of the compound of Formula I and the nuclide M.

The nuclide M of the compound of Formula II is believed to coordinate to one or more of the nitrogen atoms of the chelator and/or one or more oxygen of the carboxylic acid groups of the chelators. For instance, the nuclide M may coordinate to one or more of the nitrogen atoms of the cyclic structure and/or one or more of the carboxylic acid groups when the chelator is based on DOTA, NOTA, TETA, DTPA, NOTAGA or DOTAGA

The nuclide M may be as described herein. When M is a radionuclide, it may one of the following: ⁶⁸Ga, ¹⁸F, ⁶⁴Cu, ⁴⁴Sc, ⁸⁹Zr, ¹¹¹In, ⁶⁷Ga, ^{99m}Tc, ¹⁷⁷Lu, ^{86/90}Y. Further, the nuclide M may be selected from the following groups:

(i)⁶⁸Ga, ¹⁸F, ⁶⁴Cu, ¹¹¹n, ^{99m}Tc, Gd, ¹⁷⁷Lu and ^{86/90}y

(ii)⁶⁸Ga, or

(iii)¹⁸F.

It will be appreciated that the nuclide M described herein may be provided as a derivative and/or complex. For instance, ¹⁸F may be provided as aluminum fluoride-18 (Al¹⁸F).

The choice of the nuclide M may depend on the chelator C in the compound of Formula I.

For instance, there is provided a compound as described herein wherein:
the chelator C is DOTA and the nuclide M is ⁶⁸Ga, ⁶⁴Cu, ¹¹¹In, Mn or Gd or ¹⁷⁷Lu,
the chelator C is NOTA and the nuclide M is ⁶⁸Ga, ¹⁸F, ⁶⁴Cu or ¹¹¹In,
the chelator C is TETA and the nuclide M is ⁶⁴Cu,
the chelator C is DFO and the nuclide M is ⁸⁹Zr,
the chelator C is DTPA and the nuclide M is ¹¹¹In or ^{99m}Tc,
the chelator C is NOTAGA and the nuclide M is ⁶⁸Ga, ⁶⁴Cu or ¹¹¹In, and
the chelator C is DOTAGA and the nuclide M is ¹¹¹In, ¹⁷⁷Lu, ^{86/90}Y.

The presence of the nuclide M in the compound of Formula II described herein allows for diagnosing and/or monitoring of fibrosis. Thus, the compound of Formula II may be seen as a tracer. If the nuclide is a radionuclide, i.e. an unstable atom that may emit excess energy such as in the form of ionizing radiation, the compound of Formula II may be seen as a radiotracer. The nuclide such as the radionuclide allows for tracing the compound of Formula II when it binds to fibrotic tissue including collagen I. If the tracer is a radiotracer its radioactive decay may be used for the tracing.

The tracer described herein may be considered an imaging agent. Thus, the compound of Formula II or a pharmaceutically acceptable salt thereof may be an imaging agent. Further, the composition described herein may be an imaging agent.

The composition described herein may be a pharmaceutical composition optionally further comprising a pharmaceutically acceptable carrier, excipient and/or diluent.

There is also provided

a composition as described herein,

or

a compound of Formula II as described herein, or a pharmaceutically acceptable salt thereof

for use in diagnosing and/or monitoring of fibrosis. The diagnosing and/or monitoring may take place in a patient suffering from, suspected to be suffering from and/or being treated for, fibrosis.

There is also provided

a composition as described herein,

or

a compound of Formula II as described herein, or a pharmaceutically acceptable salt thereof

for the manufacture of a preparation for the diagnosis and/or monitoring of fibrosis.

There is also provided a use of

a composition as described herein,

or

a compound of Formula II as described herein, or a pharmaceutically acceptable salt thereof

for diagnosing and/or monitoring of fibrosis such as diagnosing and/or monitoring fibrosis in a patient suffering from, suspected to be suffering from and/or being treated for, fibrosis.

Unexpectedly, the compositions and compounds described herein have been found to allow for diagnosing and/or monitoring of fibrosis. The diagnosing and/or monitoring may involve imaging. For instance, the imaging method may be one or more of the following: Positron Emission Tomography (PET),

Single-Photon Emission Computed Tomography (SPECT), or

Magnetic Resonance Imaging (MRI).

The imaging may take place ex vivo, and/or in vivo such as in a patient.

Further, it has surprisingly been found that the compositions and compounds described herein provide good biodistribution with respect to the organs affected by the fibrosis. Good biodistribution has in particular been found for kidney fibrosis.

The choice of imaging method will influence which nuclide M is used in the compound of Formula II described herein. For instance, when PET is used as imaging method the nuclide may be ⁶⁸Ga, ¹⁸F, ⁶⁴Cu, ⁴⁴Sc, ⁸⁹Zr and ⁸⁶Y. In a further example, when SPECT is used as imaging method the nuclide M may be ¹¹¹In, ⁶⁷Ga, ^{99m}Tc, ⁹⁰Y and ¹⁷⁷Lu. In still a further example, when MRI is used as imaging method the nuclide M may be Mn or Gd.

The fibrosis described herein may be one or more of the following: liver fibrosis, kidney fibrosis, heart fibrosis, pancreas fibrosis, brain fibrosis, lung fibrosis. For instance, the fibrosis may be one or more of the following: liver fibrosis, kidney fibrosis, heart fibrosis, pancreas fibrosis, brain fibrosis, lung fibrosis such as idiopathic pulmonary fibrosis. In an example, the fibrosis may be kidney fibrosis. In particular, the fibrosis described herein may be fibrosis taking place in a solid organ such as the brain, heart, kidney, liver, lungs and pancreas. As used herein, a solid organ is an organ that has firm tissue consistency and is neither hollow nor liquid. It is also appreciated that the fibrosis mentioned herein may be fibrosis in the eye, i.e. ocular fibrosis.

Further, the diagnosing and/or monitoring of fibrosis may involve diagnosing and/or monitoring of the extent of fibrosis. For instance, the diagnosis and/or monitoring of the fibrosis my take place in conjunction with treatment of fibrosis in a patient. In this way, the usefulness of the treatment method and/or the extent of fibrosis may be assessed.

Further, there is provided a method for the diagnosis and/or monitoring of fibrosis, said method comprising the steps of:
a) administering an imaging agent selected from one or more of the following:
   a composition as described herein,
   a compound of Formula II as described herein,
   a pharmaceutically acceptable salt of a compound of Formula II as described herein,
   to a patient suffering from, suspected to be suffering from and/or being treated for, fibrosis;
b) subjecting the patient to a medical imaging technique, such as Positron Emission Tomography (PET), Single-Photon Emission Computed Tomography (SPECT) or Magnetic Resonance Imaging (MRI) imaging, and recording signals from the imaging agent administered in step a);
c) determining and/or monitoring if the patient suffers from fibrosis, and
d) optionally determining the extent of the fibrosis.

It will be appreciated that the monitoring described herein may involve monitoring the extent to which fibrosis has taken place. In this way, the progression of the fibrosis may be monitored and/or the extent of the fibrosis taking place in different patients may be monitored.

Additionally or alternatively, there is provided a method for the diagnosis and/or monitoring of fibrosis comprising the steps of:
a) subjecting a patient suffering from, suspected to be suffering from and/or being treated for, fibrosis, wherein said patient comprises a compound as described herein such as a compound of Formula II, to a medical imaging technique, such as Positron Emission Tomography (PET), Single-Photon Emission Computed Tomography (SPECT) or Magnetic Resonance Imaging (MRI) imaging, and recording signals from the radionuclide and
b) determining and/or monitoring if the patient suffers from fibrosis.

The fibrosis mentioned in the method for the diagnosis and/or monitoring of fibrosis described herein may be fibrosis as described herein.

Treatment Methods of Fibrosis

The diagnosing and/or monitoring of fibrosis described herein may be used in conjunction with a treatment method for fibrosis such as a treatment described herein. The extent of fibrosis in a patient undergoing the treatment for fibrosis may then be monitored using a composition and/or compound as described herein.

Below is a listing of some of the major organs affected by fibrosis and the treatment options currently available.

Interstitial lung disease (ILD)—includes a wide range of distinct disorders in which pulmonary inflammation and fibrosis are the final common pathways of pathology. Idiopathic pulmonary fibrosis is the most common type of ILD. ILD is usually initially treated with a corticosteroid (e.g. prednisone), sometimes in combination with drugs that supress the immune system.

Liver cirrhosis—viral hepatitis, schistosomiasis and chronic alcoholism are the main causes worldwide, but liver cirrhosis can also be developed from states of fatty-liver disease (NAFLD (non-alcoholic fatty liver disease) and NASH (non-alcoholic steatohepatitis). Treatment mainly focuses on slowing down the cause of the cirrhosis (anti-virals, diet, exercise, better diabetes control). In severe cases, a liver transplant may be required.

Chronic Kidney Disease (CKD)—is a not uncommon complication of diabetes leading to progressive loss of renal function. Untreated hypertensive diseases can also contribute. The disease is most often monitored by measuring GFR and albuminuria. Clinical management involves blood-pressure management, ARB (angiotensin-receptor blockade) or ACE-I (angiotensin-converting enzyme inhibitor), reduced sodium intake, good diabetes control, smoke cessation etc.

Heart disease—Myocardial fibrosis is a major determinant of diastolic dysfunction or failure. Diagnosis can in some cases be done by biopsy, but most often this is not feasible. Current non-invasive detection methods rely on cardiac magnetic resonance imaging and serum markers. Approved treatments include beta-blockers, ACE inhibitors, and aldosterone antagonists. Efforts to develop novel therapeutics are ongoing, targeting collagen synthesis and cross-linking.

Diseases of the eye—macular degeneration and retinal and vitreal retinopathy. Novel treatment options include VEGF-inhibitors (i.e. inhibitors of vascular endothelial growth factor) to inhibit neovascularisation in the eye.

Although the present disclosure is primarily aimed at improving the diagnosis and/or determining the extent of fibrotic disease, radiolabelling with a therapeutic isotope could potentially incur clinical benefit over currently available therapies.

The present disclosure also provides a method for the diagnosis and/or monitoring of fibrosis as described herein, wherein the patient undergoes treatment for fibrosis such as treatment involving one or more of the following: a corticosteroid, an antiviral drug, a diabetes drug, a blood pressure regulating drug, an angiotensin receptor blockade drug, an angiotensin-converting enzyme inhibitor, a beta blocker, an aldosterone antagonist, a vascular endothelial growth factor inhibitor.

Pharmaceutically Acceptable Salts

Compounds of the present disclosure may be provided in any form suitable for the intended administration. Suitable forms include pharmaceutically (i.e. physiologically) acceptable salts of a compound as disclosed herein. As used herein “pharmaceutically acceptable salt”, where such salts are possible, includes salts prepared from pharmaceutically acceptable non-toxic acids, i.e. pharmaceutically acceptable acid addition salts, or salts prepared from a base, i.e. pharmaceutically acceptable base addition salt.

Examples of pharmaceutically acceptable salts include, without limitation, non-toxic inorganic and organic acid addition salts such as hydrochloride, hydrobromide, borate, nitrate, perchlorate, phosphate, sulphate, formate, acetate, aconate, ascorbate, benzenesulphonate, benzoate, cinnamate, citrate, embonate, enantate, fumarate, glutamate, glycolate, lactate, maleate, malonate, mandelate, methanesulphonate, naphthalene-2-sulphonate, phthalate, propionate, salicylate, sorbate, stearate, succinate, tartrate, toluene-p-sulphonate, and the like. Hemisalts of acids may also be formed, for example, hemisulphate. Such salts may be formed by procedures well known and described in the art.

Other acids such as oxalic acid and trifluoroacetic acid, which may not be considered pharmaceutically acceptable, may be useful in the preparation of salts useful as intermediates in obtaining a compound of the present disclosure and its pharmaceutically acceptable acid addition salt. Most peptides of Formula I are available as trifluoroacetates. Precursors of the Formula I are heated with nuclide and thereafter eluted from a column with HCl solution. Since tracer doses are quite low when administered to a subject, any residual trifluoroacetate remaining in the tracer composition will not be harmful, thus acceptable.

Further, the pharmaceutically acceptable salt may be a base addition salt. The base addition salt may be formed from a compound of Formula I and a metal, such as an alkali metal or an alkaline earth metal. The metal may be a metal ion such as Na⁺, K⁺, Mg²⁺ or Ca²⁺. Alternatively, the salt may be formed from a compound of Formula I and an amine such as an organic amine. The amine may be ammonia, N,N′-dibenzylethylenediamine, chloroprocaine, choline, diethanolamine, ethylenediamine, N-methyl-D-glucamine or procaine.

Isomers

It will be appreciated by those skilled in the art that compounds disclosed herein may exist in stereoisomeric form(s) such as in the form of an enantiomer or a diastereoisomer. Compounds of the present disclosure include all such enantiomers, racemic mixtures thereof as well as mixtures in different proportions of the separate enantiomers. For example, there is provided a compound as disclosed herein in the form of a (−)-enantiomer or in the form of a (+)-enantiomer.

Derivatives

The present disclosure also provides a derivative of the compounds disclosed herein. The derivative may be a compound as disclosed herein wherein the chelator has been modified. For instance, one or more of the carboxylic acid groups of the chelator may be converted into e.g. an ester group or an amide group

Methods of Preparation

The compound of Formula I as described herein may be prepared as follows. Standard solid-phase peptide synthesis (SPPS) may be used to prepare the peptide Q. The resulting peptide Q may contain one or more protecting groups such as Fmoc, Trt, Pbf etc. which may be removed when appropriate. For instance, the N-terminal amino group of the peptide Q may be protected with e.g. a Fmoc group which may be removed prior to reaction with the chelator C or the linker L as described below.

The N-terminal amino group of the peptide Q may be coupled to the chelator C using a coupling reagent such as PyBOP, HBTU, Oxyma, etc. resulting in the compound C-Q.

Alternatively, the peptide Q may be coupled via its N-terminal group to the linker L to provide the compound L-Q, followed by further linking of L-Q to the chelator C to provide the compound C-L-Q. The coupling reactions may involve use of a coupling reagent such as PyBOP, HBTU, Oxyma, etc.

The compound C-Q or C-L-Q may subsequently be subjected to conditions allowing for removal of any protective groups present such as protective groups attached to one or more of the amino acids in the peptide Q.

The compound of Formula II may be obtained by combining the compound of Formula I with a nuclide M or a salt thereof as described herein. The compound of Formula I may then serve as an intermediate in the formation of the compound of Formula II.

Thus, there is provided a method for preparing a compound of Formula II as described herein, said method comprising the steps of:
a) preparing a compound of Formula I as described herein, and
b) combining the compound of Formula I with a nuclide M as described herein thereby providing the compound of Formula II.

The compound of Formula I and the nuclide M may be combined in equimolar amounts to provide a compound of Formula II in which the ratio between the compound of Formula I and the nuclide 1 is equal to one, i.e. 1/1. However, it may not always be possible to control the stoichiometry and therefore the compound of Formula I and the nuclide M may be combined in unequal amounts, such as unequal molar amounts, resulting in a composition comprising the aforementioned compound of Formula II, in which the ratio between the compound of Formula I and the nuclide is one, and an additional amount of the compound of Formula I and/or nuclide M.

It will be appreciated that the nuclide M may be a radionuclide produced using a radionuclide generator or a cyclotron as known in the art.

### ABBREVIATIONS

ACE-I Angiotensin Converting Enzyme Inhibitor
ARB Angiotensin Receptor Blockade
BALB/c BALB/c is an albino, laboratory-bred strain of the house mouse
BSA Bovine Serum Albumin
Bq Becquerel
CBP8 Collagen Binding Peptide 8
cc Cubic Centimeter
CKD Chronic Kidney Disease
DCM Dichloromethane
DFO Desferrioxamine B
DMF Dimethylformamide
DIEA N, N-Diisopropylethylamine
DOTA 1,4,7,10-Tetraazacyclododecane-1,4,7,10-tetraacetic acid
DOTAGA 2-[4,7,10-Tris(carboxymethyl)-1,4,7,10-tetraza-1-cyclododecyl]glutaric acid
DTPA Diethylenetriaminepentaacetic acid
E Glutamic acid (Glu)
ESI Electrospray Ionization
Fmoc Fluorenylmethyloxycarbonyl protecting group
g gram(s)
GFR Glomerular Filtration Rate
H Histidine (His)
HBTU (2-(1H-benzotriazol-1-yl)-1,1,3,3-tetramethyluronium hexafluorophosphate
HPLC High Performance Liquid Chromatography
ILD Interstitial lung disease
ivDde 4,4-dimethyl-2,6-dioxocyclohex-1-ylidene)isovaleryl
L Leucine (Leu)
MBq Mega Becquerel
min. minute(s)
MRE Magnetic Resonance Elastography
MRI Magnetic Resonance Imaging
MS Mass Spectroscopy
N Asparagine (Asn)
NAFLD Non-Alcoholic Fatty Liver Disease
NASH Non-Alcoholic Steatohepatitis)
NNM N-Methylmorpholine
NOTAGA 1,4,7-Triazacyclononane-1-glutaric acid-4,7-acetic acid
NOTA 1,4,7-Triazacyclononane-1,4,7-triacetic acid
nM nanomolar
Pbf 2,2,4,6,7-Pentamethyldlhydrobenzofuran-5-sulfonyl
PBS Phosphate-buffered saline
PET Positron Emission Tomography
p.i. post injection
PyBOP Benzotriazol-1-yl-oxytripyrrolidinophosphonium hexafluorophosphate
R Arginine (Arg)
ROI Regions of interest
RP-HPLC Reversed phase high performance liquid chromatography
SPECT Single-Photon Emission Computed Tomography
SPPS Solid Phase Peptide Synthesis
SPR Surface Plasmon Resonance
SUV Standardized Uptake Value
t-Bu tert-Butyl
TES Triethylsilane
TETA 1,4,8,11-Tetraazacyclotetradecane-1,4,8,11-tetraacetic acid
TFA Trifluoroacetic acid
Trt Trityl
UV Ultraviolet
VEGF Vascular Endothelial Growth Factor

### Material and Methods

Materials

The purchased chemicals were used without further purification: amino acids (Novabiochem, Switzerland, Sigma-Aldrich, Sweden, Iris Biotech GmbH, Germany), PyBOP (Novabiochem, Switzerland), 2CTCresin (Iris Biotech GmbH, Germany), Fmoc-O2Oc—OH (Iris Biotech GmbH, Germany), DOTA(tBu)3-OH and NOTA(tBu)2-OH (CheMatech, France), piperidine (Sigma-Aldrich, Sweden), DMF (Fisher Scientific, UK), sodium acetate buffer (pH 4.6, 31048, Sigma-Aldrich, Stockholm, Sweden), 30% HCl (Ultrapure, 1.00318.0250 Merck, Sigma-Aldrich) and trifluoroacetic acid (TFA, Merck, Darmstadt, Germany). Some of the compounds were prepared by external labs (Vivitide/NEP)

### Peptide Synthesis and Coupling of the Linker and Chelator (Method A)

This method is here exemplified for synthesis of compounds 1 and 16 below. Standard solid-phase peptide synthesis (SPPS) was used to synthesize the precursor peptides by conjugating 2-(4,7,10-tris(2-(tert-butoxy)-2-oxoethyl)-1,4,7,10-tetraazacyclododecan-1-yl)acetic acid (DOTA(tBu)₃) or 2-(4,7,10-tris(2-(tert-butoxy)-2-oxoethyl)-1,4,7-triazacyclononane-1,4,7-triacetic acid (NOTA(tBu)₃) to the peptide sequence LRELHLNNN via a linker (—X—(CH₂CH₂O)₂—CH₂—C(O)—). All reactions were performed at room temperature unless otherwise noted.

Fmoc-Asn(Trt)-OH (238.7 mg, 0.40 mmol) and diisopropylethylamine (DIEA) in 6.0 mL dry dichloromethane (DCM) was added to 2-chlorotrityl resin (375 mg, loading 1.6 mmol/g). After 2 h 0.30 mL MeOH was added and reacted for 15 min. The resin was washed with DMF (2×5 mL) and DCM (2×5 mL), dried in vacuum to give 584.5 mg Fmoc-Asn(Trt) bound resin. New loading was calculated to 0.64 mmol/g and the side chain protected peptide LRELHLNNN was synthesized in a 4 mL disposable syringe equipped with a porous polyethylene filter on a 374 μmol scale using SPPS and Fmoc/tert-butyl (tBu) protection. For the Fmoc protected amino acids the side chain protection were as follows: Asn(Trt), Arg(Pbf), Glu(Ot-Bu), His(Trt). 20% Piperidine in DMF (4×2 mL) was used to remove the Fmoc group after each coupling step and the amino acids were coupled overnight using PyBOP (540 μmol) in DMF (2 mL) in presence of DIEA (800 μmol). After completion of the coupling steps, the partially protected peptide on resin was washed with several portions of DMF, DCM and MeOH and dried in vacuum.

Part of the peptide on resin (approximately 30 μmol) was transferred to a 2 mL disposable syringe equipped with a porous polyethylene filter and after deprotection of the Fmoc-group coupled for 21 h with Fmoc-NH—(CH₂CH₂O)₂—CH₂—C(O)—OH, 2 equivalents) using PyBOP (2 equivalents) and DIEA (3 equivalents) in 0.5 mL DMF. The Fmoc group was removed by treatment with 20% piperidine in DMF (2 mL for 1 min+3×2 mL for 10 min). After washing of the resin, DOTA(tBu)₃-OH (2 equivalents) or NOTA(tBu)₃-OH (2 equivalents) were coupled for 20 h using PyBOP, and DIEA DMF. The resins were then washed extensively with DMF and DCM and dried in vacuum.

The resins were transferred to a centrifuge tube and treated with triethylsilane (TES) and 95% aqueous TFA and the mixture was rotated for 2 h. The resins were removed by filtration and washed with TFA. The filtrates were partly evaporated under a stream of nitrogen and the crude products were precipitated by addition of diethyl ether. The precipitates were collected by centrifugation, washed with diethyl ether and dried in vacuum.

The crude, deprotected products were dissolved in 10% acetonitrile in water and purified with preparative reversed high-performance liquid chromatography (RP-HPLC). The preparative column used was a Nucleodur C18 HTec (21×125 mm, particle size 5 μm) and eluent was a CH₃CN/H₂O gradient with 0.1% TFA at a flow rate of 10 mL/min and with UV detection at 220 nm. The pure fractions were lyophilized and the two products were obtained with more than 98% purity determined from the 214 nm trace in a HPLC run.

Analytical RP-HPLC was performed on a Dionex UltiMate 3000 HPLC system using a Penomenex Kinetex C18 column (50×3.0 mm, 2.6 μm particle size, 100 Åpore size). A gradient of H₂O/CH₃CN/0.05% HCOOH was used as eluent at a flow rate of 1.5 mL/min. For detection UV and a Bruker amazon SL ion trap mass spectrometer with electrospray ionization (ESI) MS with positive mode scanning was used. The mass spectrometry analysis detected m/z=827.5 for [M+2H]²⁺, 551.8 for [M+3H]³⁺ and m/z=414.4 for [M+4H]⁴⁺, with reconstituted molecular weight of 1652.85 for Compound 1,DOTA-NH—(CH₂CH₂O)₂—CH₂—C(O)-LRELHLNNN—OH; and m/z=776.8 for [M+2H]²⁺ and 518.3 for [M+3H]³⁺, with reconstituted molecular weight of 1551.8 for Compound 16, NOTA-NH—(CH₂CH₂O)₂—CH₂—C(O)-LRELHLNNN—OH.

### Alternative Procedure for Peptide Synthesis and Coupling of the Linker and Chelator (Method B)

The peptides of the invention can be synthesized using standard solid phase peptide chemistry with FMOC protected amino acids on resin using an automated synthesizer (e.g. AMS 422 Multiple Peptide Synthesizer or CEM Liberty Blue). Fmoc-protected amino acids are commercially available from sources as indicated above. For C-terminal amides RINK resins were used, e.g. Novabiochem Rink Amide AM Resin (200-400 mesh), loading 0.64 mml/g, whereas for C-terminal acids preloaded Wang resins (100-200 mesh), loading 0.50 mmol/g were used. Amino acid activation and couplings are carried out with HBTU (typically 6 equivalents) and NMM (N-methylmorpholine, typically 12 equivalents). FMOC groups are removed using 20% piperidine in DMF. When the linker-chelator is attached to the N-terminus, the linker Fmoc-NH—(CH₂CH₂O)₂—CH₂—C(O)—OH (2 eq.) is coupled manually after removal of the Fmoc-group of the last amino-acid (e.g. leucine) of the peptide sequence using a standard activation procedure (HBTU/2M DIEA as activator/base) at 40° C. for 3 h. To ensure complete coupling that step is repeated. Complete coupling can be monitored by applying the Kaiser test. The Fmoc-group of the linker is removed by using 20% piperidine in DMF. Finally, 2-(4,7,10-tris(2-(tert-butoxy)-2-oxoethyl)-1,4,7,10-tetraazacyclododecan-1-yl)acetic acid (DOTA(tBu)₃) is coupled to the free N-terminal amino group by a standard amino acid activation procedure (double-coupling, 2 equivalents of (DOTA(tBu)₃, HBTU/2M DIEA as activator & base, 40° C., 3 h). The resin-bound sequence is then cleaved using a cocktail of TFA/water/thioanisole/ethylmethylsulfide/ethanedithiol (20 ml: 1 ml: 1 ml: 1 ml: 1 ml).

Peptides are precipitated in ether/hexane and then isolated by centrifugation. The dried peptide pellets are reconstituted in a water and acetonitrile mixture and lyophilized. The lyophilized raw product is purified by preparative reverse phase HPLC (10 μm C18 column, 25×250 mm) with acetonitrile-water buffers containing 0.1% TFA as eluent. Peptide containing fractions are analyzed and pure fractions are pooled and lyophilized. Analytical HPLC data is obtained on a 2.6 μm C18 analytical column with water-acetonitrile gradients containing 0.1% TFA as eluent. Molecular weight is confirmed by MS analysis using a Bruker amaZon SL instrument. Compounds 2-6, 11, and 12 in Example 2 below were synthesized according to Method B.

### Alternative Procedure for Peptide Synthesis (Method C), Used for Cases when the Linker and Chelator are Coupled to an Amino Functionality within an Amino Acid Side Chain

In this case, the peptides of the invention can be synthesized using a protocol very similar to method B but using a special protected amino acid which allows selective coupling to the amino function of the amino acid side chain. Peptide assembly is accomplished by standard solid phase peptide chemistry with FMOC protected amino acids on resin using an automated synthesizer (e.g. AMS 422 Multiple Peptide Synthesizer or CEM Liberty Blue). Fmoc-protected amino acids are commercially available from sources as indicated above. For side chain modification Fmoc protected amino acids are used, in which the sidechain, e.g. the lysine, is protected by an orthogonally cleavable protecting group such as Fmoc-Lys(ivDde)-OH. For C-terminal amides RINK resins were used, e.g. Novabiochem Rink Amide AM Resin (200-400 mesh). Amino acid activation and couplings are carried out with HBTU (typically 6 equivalents) and NMM (N-methylmorpholine, typically 12 equivalents). FMOC groups are removed using 20% piperidine in DMF. After assembly of the peptide on solid phase, the N-terminal Fmoc group is removed using 20% piperidine in DMF, and the N-terminus is protected by using Boc-anhydride. The ivDde protecting group on the amino acid to be functionalized can then be removed by using 2% hydrazine in DMF (2×30 min). A test cleavage confirms ivDde removal. The linker, e.g. Fmoc-NH—(CH₂CH₂O)₂—CH₂—C(O)—OH (2 eq.) is coupled manually using a standard activation procedure (HBTU/2M DIEA as activator/base, 40° C., 3 h). To ensure complete coupling that step is repeated. Complete coupling can be monitored by applying the Kaiser test. The Fmoc-group of the linker is removed by using 20% piperidine in DMF. Finally, 2-(4,7,10-tris(2-(tert-butoxy)-2-oxoethyl)-1,4,7,10-tetraazacyclododecan-1-yl)acetic acid (DOTA(tBu)₃) is coupled to the free amino group by a standard amino acid activation procedure (double-coupling, 2 equivalents of (DOTA(tBu)₃, HBTU/2M DIEA as activator & base, 40° C., 3h). The resin-bound sequence is then cleaved using a cocktail of TFA/water/thioanisole/ethylmethylsulfide/ethanedithiol (20 ml: 1 ml: 1 ml: 1 ml: 1 ml). Peptides are precipitated in ether/hexane and then isolated by centrifugation. The dried peptide pellets are reconstituted in a water and acetonitrile mixture and lyophilized. The lyophilized raw product is purified by preparative reverse phase HPLC (10 μm C18 column, 25×250 mm) with acetonitrile-water buffers containing 0.1% TFA as eluent. Peptide containing fractions are analyzed and pure fractions are pooled and lyophilized. Analytical HPLC data is obtained on a 2.6 μm C18 analytical column with water-acetonitrile gradients containing 0.1% TFA as eluent. Molecular weight is confirmed by MS analysis using a Bruker amaZon SL instrument. For example, compound 13 in Example 2 below was synthesized according to Method C.

### Radiochemistry

### Gallium-68 Radiochemistry

A ⁶⁸Ga/⁶⁸Ga generator system with ⁶⁸Ge attached to a column packed with titanium dioxide (1850 MBq, Eckert & Ziegler, Eurotope GmbH) was eluted with 0.1 M HCl, in order to obtain ⁶⁸Ga (t_{1/2}=68 min, β+=89% and EC=11%). Second fraction of 1 ml containing 70-80% of the generator radioactivity was buffered with 100 μl of sodium acetate buffer (pH 7) to ensure pH 4.2-4.6. After controlling the pH, 20 nanomoles (1 mM) of Compound 1 or Compound 5 dissolved in deionized water was added, and the mixture was incubated in a heating block at 75° C. for 15 minutes. Following incubation, the crude product was left to cool down for two minutes and purified on solid phase extraction cartridge (HLB, Oasis) to obtain the pure product in 50% ethanol. Further, the product was analyzed by HPLC-UV-Radio system (VWR Hitachi Chromaster pump 5110, Knauer UV detector 40D equipped with a remote UV flow cell, Bioscan Flow count equipped with an Eckert & Ziegler extended range module Model 106 and a Bioscan B-FC-3300 radioactivity probe and a VWR Hitachi Chromaster A/D Interface box). Separation of the analytes was accomplished using analytical column (Hichrom Vydac 214MS, 5 μm C4, 50×4.6 mm). The conditions were as followed: A=0.1% TFA in H₂O; B=0.1% TFA in 70% CH₃CN, with UV-detection at 220 nm; linear gradient over 15 min, 5-70% solvent B linear gradient over 15 minutes, flow rate was 1.0 mL/min. Data acquisition and handling were performed using Agilent OpenLAB Chromaster EZChrome Edition version A.04.05.

### Aluminum Fluoride-18 Radiochemistry

¹⁸F was produced by a Scanditronix MC-17 cyclotron by proton bombardment of ¹⁸O enriched water (>97%). Typically, 3-5 GBq of radioactivity was produced. The radioactivity was transferred to a hotcell and passed through a QMA SPE cartridge to retain fluorine-18. The cartridge was washed with water (1 mL) and then the radioactivity 200 μL NaCl solution (0.9%). To a 1.5 mL vial was added 20 μL Compound 16 (40 nmol, 2 mM solution in NaOAc pH 4.6), 10 μL of AlCl₃ (2 mM in NaOAc pH 4.6), 50 μL NaOAc (pH 4.6) and 100 μL EtOH (99%). 50 μL of the saline solution containing ¹⁸F was added to vial and then it was heated to 100° C. for 15 min. The reaction mixture was diluted with water (3 mL) and added to an HLB SPE cartridge which was then washed with water (3×1 mL). The product was eluted with 400 μL of EtOH (99%) and further diluted with 3.6 mL PBS. Quality control was performed in the same manner as with ⁶⁸Ga using a gradient of 10-90% CH₃CN in 50 mM ammonium formate (AMF, pH 3.5) over 8 minutes using a Phenomenex LUNA C18. The activity yield was 0.3-0.8 GBq (10-20%, non-decay corrected).

The present disclosure is further illustrated in the following non-limitative examples.

### Example 1

Compounds 1 and 16 were synthesized according to Method A described above.

DOTA-NH—(CH₂CH₂O)₂—CH₂—C(O)-LRELHLNNN—OH Compound 1

Purity: 95%; Mass detected m/z=827.5 for [M+2H]²⁺, 551.8 for [M+3H]³⁺ and m/z=414.4 for [M+4H]⁴⁺, with reconstituted molecular weight of 1652.85 for DOTA-NH—(CH₂CH₂O)₂—CH₂—C(O)-LRELH LNNN—OH

NOTA-NH—(CH₂CH₂O)2-CH₂—C(O)-LRELHLNNN—OH Compound 16

Purity: 95%; Mass detected m/z=776.8 for [M+2H]²⁺ and 518.3 for [M+3H]³⁺, with reconstituted molecular weight of 1551.8 for NOTA-NH—(CH₂CH₂O)₂—CH₂—C(O)-LRELHLNNN—OH.

### Example 2

Compounds 2-6, 11 and 12 were synthesized according to Method B or a variation of it. Compound 13 was synthesized in accordance with Method C.

DOTA-NH—(CH₂CH₂O)₂—CH₂—C(O)-L-R-E-L-H-L-N-A-N—OH Compound 2:

Purity: 98.1%; Mass detected m/z=1610.83 (705.92 as M+2), (537.62 as M+3) theoretical molecular weight: 1610.8

DOTA-NH—(CH₂CH₂O)₂—CH₂—C(O)-L-R-E-L-H-L-N—N—N—NH₂ Compound 3

Purity: 99.1%; Mass detected m/z=1653.01 (827.02 as M+2), (551.96 as M+3) theoretical molecular weight: 1652.80

DOTA-NH—(CH₂CH₂O)₂—CH₂—C(O)-L-R-E-L-H-L-N—N-n-OH Compound 4

Purity: 89.3%; Mass detected m/z=1654.58 (827.34 as M+2), (551.90 as M+3), theoretical molecular weight: 1654.8

DOTA-NH—(CH₂CH₂O)₂—CH₂—C(O)-L-R-E-L-H-L-N-A-N—NH₂ Compound 5

Purity: 100%; Mass detected m/z=1609.8 (805.45 as M+2), (537.25 as M+3), theoretical molecular weight:1609.8

DOTA-NH—(CH₂CH₂O)₂—CH₂—C(O)-L-R-E-L-H—V-N—N—N—OH Compound 6

Purity: 98.7%; Mass detected m/z=1640.76 (820.41 as M+2), (547.28 as M+3) theoretical molecular weight: 1641.00

DOTA-NH—(CH₂CH₂O)₃—CH₂—C(O)-L-R-E-L-H-L-N—N—N—OH Compound 11

Purity: 98.7%; Mass detected m/z=1698.77 (849.43 as M+2), (566.61 as M+3) theoretical molecular weight: 1699.0

DOTA-L-R-E-L-H-L-N—N—N—OH Compound 12

Starting from Fmoc Asn(Trt)-Wang Resin (100-200 mesh), loading 0.50 mmol/g.

Purity: 99.3%; Mass detected m/z=1508.7 (754.88 as M+2), (503.58 as M+3) theoretical molecular weight: 1508.4

H₂N-L-R-E-L-H-L-N—N—N—K(DOTA-NH—(CH₂CH₂O)₂—CH₂—C(O))—NH₂ Compound 13

was synthesized according to Method C using Fmoc-Lys(ivdDe)-OH and Novabiochem Rink amide AM Resin LL (100-200 mesh)(loading 0.29 mmol/g). Standard amino acid couplings were carried out in this case with 5 equivalents DIC/Oxyma.

Purity: 95.1%; Mass detected m/z=1781.9 (890.97 as M+2), (594.35 as M+3) theoretical molecular weight: 1782.2

Likewise using Methods B or C the following peptides can be prepared:

DOTA-NH—(CH₂CH₂O)₂—CH₂—C(O)-L-R-E-1-H-L-N—N—N—OH Compound 7

DOTA-NH—(CH₂CH₂O)₂—CH₂—C(O)-L-R-E-L-H-L-N—N—OH Compound 8

DOTA-NH—(CH₂CH₂O)₂—CH₂—C(O)—H-L-R-E-L-H-L-N—N—N—OH Compound 9

DOTA-NH—(CH₂CH₂O)₂—CH₂—C(O)-L-R-E-L-H-L-N—N—N—K—OH Compound 10

H₂N-L-R-E-K(DOTA-NH—(CH₂CH₂O)₂—CH₂—C(O))—H-L-N—N—N—OH Compound 14

H₂N-L-R-E-L-H—K(DOTA-NH—(CH₂CH₂O)₂—CH₂—C(O))—N—N—N—OH Compound 15

### Example 3

Stability Testing of Peptides

Analytical high performance liquid chromatography (HPLC) was performed on a Dionex UltiMate 3000 HPLC system with a Bruker amazon SL ion trap mass spectrometer and detection by UV (diode array detector, 214, 254, and 280 nm) and electrospray ionization (ESI) MS using a Penomenex Kinetex C18 column (50×3.0 mm, 2.6 μm particle size, 100 Åpore size) and a Penomenex Kinetex Biphenyl column (50×4.6 mm, 2.6 μm particle size, 100 Åpore size). A gradient of H₂O/CH₃CN/0.05% HCOOH was used at a flow rate of 1.5 mL/min.

Method A: Detection at 214 nm

column: Penomenex Kinetex C18 (50×3.0 mm, 2.6 μm particle size, 100 Åpore size)

solvent: H₂O+0.05% HCOOH: CH₃CN+0.05% HCOOH (flow 1.5 ml/min)

gradient: 0-100% CH₃CN+0.05% HCOOH (5 min)

volume: 1 μl

mass analyzer: Bruker amaZon SL ion trap mass spectrometer, electrospray positive ion mode

Method B: Detection at 214 nm

column: Penomenex Kinetex Biphenyl column (50×4.6 mm, 2.6 μm particle size, 100 Åpore size)

solvent: H₂O+0.05% HCOOH: CH₃CN+0.05% HCOOH (flow 1.5 ml/min)

gradient: 0-100% CH₃CN+0.05% HCOOH (5 min)

volume: 1 μl

mass analyzer: Bruker amaZon SL ion trap mass spectrometer, electrospray positive ion mode

For stability testing, 500 μmol pure compound was dissolved in 1 mL PBS buffer (pH 7.4), or 1 mL sodium acetate buffer (pH 4.5, 100 mM). For the peptides stored in PBS, the solutions were stored for 14 days at 4° C. and 23° C. At 0 h, 1 day, 7 days and 14 days, the solutions were analyzed by HPLC (analytical HPLC Method A and Method B).

For the peptides stored in sodium acetate buffer (pH 4.5, 100 mM) the solutions were analyzed by HPLC (analytical HPLC Method B) after 0 h, and 1 day incubation at 4° C. and 23° C.

The “% Purity” at each time point is defined by the % Relative purity at time point “n” (n=1 day, 7 days, 14 days in pH 7.4 PBS and 1 day at pH 4.5 NaAc) in relation to the % Relative purity at t₀ following the equation:

% Purity at tₙ=[(% Relative purity tₙ)×100)]/% Relative purity t₀

The % Relative purity at to was calculated by dividing the peak area of the peptide at to by the sum of all peak areas at to following the equation:

% Relative purity t₀=[(peak area t₀)×100]/sum of all peak areas t₀

Similarly, the % relative purity tₙ was calculated by dividing the peak area of the peptide at tₙ by the sum of all peak areas at tₙ following the equation:

% Relative purity tₙ=[(peak area tₙ)×100]/sum of all peak areas tₙ

The results of the stability tests of the compound of the invention are given below in Table II, Table III and Table IV.

Table II shows the chemical stability of the peptides after incubation at pH 7.4. Samples were incubated up to 14 days at 23° C. and 4° C. and were analyzed using HPLC Method A.

TABLE II
Table Stability at pH 7.4, HPLC Method A

| Compound | | % Purity after | | |
|---|---|---|---|---|
| No | Temp | 1 day | 7 days | 14 days |

| | | | | | |
|---|---|---|---|---|---|
| 1 | 4° | C. | >99 | >99 | >99 |
| | 23° | C. | >99 | >98 | >97 |
| 2 | 4° | C. | >99 | >99 | >99 |
| | 23° | C. | >98 | >98 | >97 |
| 3 | 4° | C. | 100 | >99 | >98 |
| | 23° | C. | >97 | >97 | >96 |
| 5 | 4° | C. | >99 | >99 | >98 |
| | 23° | C. | >98 | >97 | >96 |
| 12 | 4° | C. | >99 | >99 | |
| | 23° | C. | >99 | >96 | |
| 13 | 4° | C. | 100 | 100 | |
| | 23° | C. | 100 | 100 | |
| 6 | 4° | C. | >99 | >99 | |
| | 23° | C. | 100 | >99 | |
| 11 | 4° | C. | >99 | >97 | |
| | 23° | C. | >99 | >96 | |

Table III shows the chemical stability of the peptides after incubation at pH 7.4. Samples were incubated up to 14 days at 23° C. and 4° C. and were analyzed using HPLC Method B.

TABLE III
Stability at pH 7.4, HPLC Method B

| Compound | | % Purity after | | |
|---|---|---|---|---|
| No | Temp | 1 day | 7 days | 14 days |

| | | | | | |
|---|---|---|---|---|---|
| 1 | 4° | C. | 100 | >98 | >96 |
| | 23° | C. | >99 | >98 | >97 |
| 2 | 4° | C. | 100 | >98 | >99 |
| | 23° | C. | 100 | >99 | >98 |
| 3 | 4° | C. | >99 | >98 | >96 |
| | 23° | C. | >99 | >98 | >98 |
| 5 | 4° | C. | >99 | >99 | >98 |
| | 23° | C. | 100 | >99 | >98 |
| 12 | 4° | C. | >99 | >99 | |
| | 23° | C. | >99 | >95 | |
| 13 | 4° | C. | >99 | >99 | |
| | 23° | C. | >99 | >99 | |
| 6 | 4° | C. | >99 | >99 | |
| | 23° | C. | >99 | >99 | |
| 11 | 4° | C. | >99 | >99 | |
| | 23° | C. | >99 | >99 | |

Table IV shows the chemical stability of the peptides after incubation at pH 4.5. Samples were incubated for 1 day at 23° C. and 4° C. and were analyzed using HPLC Method B.

TABLE IV
Stability at pH 4.5, HPLC Method B

| | | |
|---|---|---|
| Compound No | Temp | % Purity after 1 day |

| | | | |
|---|---|---|---|
| 1 | 4° | C. | >95 |
| | 23° | C. | >96 |
| 2 | 4° | C. | >99 |
| | 23° | C. | >99 |
| 3 | 4° | C. | >99 |
| | 23° | C. | >99 |
| 5 | 4° | C. | >99 |
| | 23° | C. | >99 |
| 12 | 4° | C. | >98 |
| | 23° | C. | >98 |
| 13 | 4° | C. | >99 |
| | 23° | C. | >99 |
| 6 | 4° | C. | >99 |
| | 23° | C. | >99 |
| 11 | 4° | C. | >99 |
| | 23° | C. | >98 |

### Example 4

Radiolabelling

Compound 1 was labelled with ⁶⁸Ga (n=7) and purified using a solid-phase extraction cartridge, resulting in a radiochemical purity of >97%.

Compound 16 was labelled with Al¹⁸F (n=5) and purified using a solid-phase extraction cartridge, resulting in a radiochemical purity of >99%

Compound 5 was labelled with ⁶⁸Ga (n=3) and purified using a solid-phase extraction cartridge, resulting in a radiochemical purity of >99%

### Example 5

In Vitro Autoradiography Binding Assay on Tissue Sections

Frozen liver from mice (female, Balb/c, Taconic) with various grade of fibrosis (Treatment with 0.5 mg CCl₄/g body weight i.p.3 times per week for 3 weeks), as well as control livers (female, Balb/c, Taconic), were sectioned to 20 μm sections with a cryostat microtome (Micron HM560, Germany), mounted on Menzel Super Frost plus glass slides, dried at room temperature (RT) and stored at −20° C. until used in the study. The sections were pre-incubated for 10 minutes at RT in PBS buffer containing 1% BSA (to reduce tracer binding to the glass surface). Further, the sections were incubated at 200 nM (approximately at the expected K_{d} of 170 nM) concentration of [⁶⁸Ga]Ga-DOTA-NH—(CH₂CH₂O)₂—CH₂—C(O)-LRELHLNNN—OH ([⁶⁸Ga]Ga-1) for 40 minutes at RT in order to determine the total binding of the tracer. To determine the non-specific binding of the tracer, section duplicates were incubated in the presence of 60 μM unconjugated peptide, i.e. LRELHLNNN. Following the incubation with the tracer, the sections were washed one minute in ice-cold PBS containing 1% BSA, and two times, one minute each in ice-cold PBS. Further, the sections were dried under a stream of warm air (37° C.) for 10 min. As a reference, 20 μl of the incubation solution was applied to a filter paper. The sections together with the reference were exposed to phosphor imaging plates for 2.5 h, and scanned by a Phosphorimager system (Cyclone Plus, Perkin Elmer). The sections were visualised and analysed using the software ImageJ (ImageJ 1.45S, NIH, Bethesda, USA). Regions of interest (ROIs) were drawn on the liver tissues in the image, and the mean values of the tissue ROIs were corrected for background uptake. Specific binding was defined as the difference between total binding and non-displaceable binding, and the percentage of specific binding was defined as the ratio between the specific binding and the total binding multiplied by 100. Separate sections from the same biopsy were stained with Sirius Red to assess the grade of fibrosis.

The uptake of [⁶⁸Ga]Ga-1 on the frozen sections of fibrotic mice liver was inhibited using 60 μM of unconjugated LRELHLNNN peptide (FIG. 9a). No detectable blocking effect was observed in healthy controls without fibrosis, as expected. [⁶⁸Ga]Ga-1 demonstrated a significant correlation (p<0.05) in binding (in the range of 1-80 fmol/mm³) to grade 0-3 fibrotic liver tissue (n=10), with a correlation coefficient of 0.4 (FIG. 9b). The binding to healthy liver tissue controls (n=2) was in the range of 2-22 fmol/mm³.

### Example 6

Surface Plasmon Resonance Assay was Used to Measure Interactions with Collagen Type 1

Surface plasmon resonance (SPR) binding analysis was performed with a Biacore 3000 instrument (Cytiva). 0.3 g/L Purecol bovine collagen type I in 10 mM sodium acetate pH 4.2 was injected over an NHS/EDC activated CM5 sensor surface (Cytiva) to a response of nearly 10000 RU. In parallel, a blank surface was prepared by activation by NHS/EDC. Both surfaces were passivated using 1 M ethanolamine pH 8.5.

Serial dilutions of Q-peptides from 100 μM to 12.5 μM diluted in 1× HBS-EP (Cytiva) were injected over the surfaces sequentially with blank injections of buffer between every distinct peptide, with 1 minute association and disassociation time for each sample.

### Example 7

Ligand-Tracer Technology was Used to Measure Interactions with Collagen Type 1

Kinetics of tracer binding to and dissociation from collagen type 1 can also be measured in real-time using Ligand-Tracer Yellow instruments (Ridgeview Instruments AB) at RT. Corning CellBIND cell culture dishes (100 mm) will be partially coated with collagen (500 ug/mL in 0.02M acetic acid). The dishes will be incubated overnight at 37° C., then excess collagen will be removed, and the surface will be washed with 10 mL 1% BSA/PBS solution. Uptake curves will be measured at increasing concentrations of ⁶⁸Ga-labeled peptides, then the medium will be replaced by fresh medium in order to follow the dissociation. Association rate, dissociation rate and equilibrium dissociation constant will be calculated using TraceDrawer software (Ridgeview Instruments AB).

### Example 8

Ex Vivo Organ Distribution in Healthy Rats

Sprague Dawley rats (obtained from Taconic, n=22, male, healthy, weight 287±25 g) were used for ex vivo organ distribution assessment of biodistribution and dosimetry.

Five MBq of [⁶⁸Ga]Ga-1 (n=10) (corresponding to 5-10 μg) in phosphate-buffered saline (PBS, pH 7.4) was injected intravenously as a bolus to conscious rats. The animals were euthanized by a CO₂—O₂ mixture 10, 20, 40, 60 and 120 minutes post-injection. The radioactivity of the excised organs was measured in a gamma counter. Samples from blood, heart, lung, liver, spleen, adrenal glands, kidneys, intestines, with or without contents, muscle, testis, bone, brain, pancreas, urine bladder and bone marrow were collected. The remaining carcass was also measured in order to monitor the radioactivity elimination and recovery. The radioactivity readings were decay-corrected to the time of the injection, and the results were expressed as standardized uptake values (SUV).

### Experiment 9

In Vivo Biodistribution in Healthy Rats by PET/MRI Imaging

Biodistribution was measured by PET/MRI imaging in additional rats using a small animal PET-MRI system (nanoPET/MRI, 3T magnet, Mediso, Hungary). Anaesthetized animals were administered 5 MBq [⁶⁸Ga]Ga-1 (n=5) or [⁶⁸Ga]Ga-5 (n=3) via the tail vein. Dynamic whole-body PET scanning for up to 150 minutes was performed using multiple whole-body sweeps (3 beds per passage; 2×5 min, 2×10 min, 4×30 min). Anatomical axial and coronal MR images were measured with T1-weighted (T1W) spin echo sequences. PET images were reconstructed by the use of Maximum Likelihood Estimation Maximized (MLEM) algorithm (10 iterations). Maximum Intensity Projection (MIP) images were generated in Carimas 2.9 (Turku PET Center, Turku, Finland) to allow quantitative visualization of radiotracer uptake distribution in the entire body.

### Example 10

Extrapolation of the Predicted Human Dosimetry from Rat Biodistribution Data

Data from dynamic biodistribution data on healthy rats was used to calculate the human predicted dosimetry. The residence times were calculated using trapezoidal model approximation of the organ uptake values (un-decay corrected) extrapolated to a model of human tissues weights. For dose assessment, OLINDA/EXM 1.1 software was used to compute the absorbed human doses in various organs on male phantoms.

### Example 11

Results of Biodistribution and Dosimetry Calculations

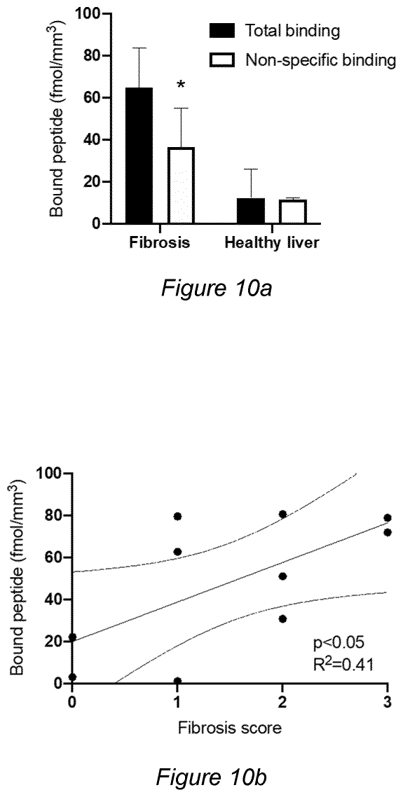

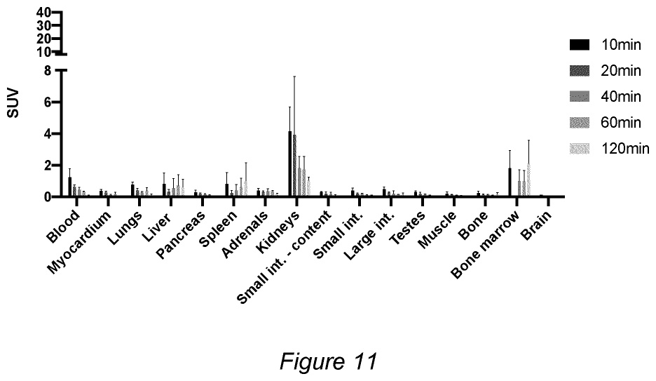

Ex vivo organ distribution data from 19 organs was presented as decay-corrected SUV values. [⁶⁸Ga]Ga-1 revealed fast blood clearance and washout from most of the organs with SUV values below one (FIG. 10). The kidney SUV was at the level of four at the 10-minute time point, with a decrease to SUV˜1 after 120 min p.i., indicating on fast renal excretion and low renal trapping. The pattern of the biodistribution was the same as assessed by dynamic PET (n=3). Red bone marrow exhibited the highest organ absorbed dose of 0.033 mSv/MBq thus being the critical organ. The total effective dose for [⁶⁸Ga]Ga-1 was 13 μSv/MBq. The effective dose allows administration of up to 770 MBq to humans annually; corresponding to at least three PET scans of 200 MBq.

### Example 12

Induction of Lung Fibrosis by Bleomycin Administration in Rat

Bleomycin was administered intratracheally in lightly sedated rats (1500 units in 200 μl saline). The health of the animals was followed for up to 2 weeks, when PET examinations were performed.

### Example 13

In Vivo Binding in a Model of Bleomycin Induced Lung Fibrosis

Compound 1 or Compound 5 labelled with Gallium-68 was administered to rats with bleomycin induced lung fibrosis or control rats without induced fibrosis. The animals were examined for binding in lung, as well as other tissues, by in vivo PET scanning and/or ex vivo organ distribution and measurement in a gamma counter. After gamma counter measurement, lung tissues were immediately frozen and embedded in OCT medium. The embedded tissue was cryo-sectioned, and the sections exposed to a phosphor-imager plate to visualize the tissue binding distribution.

Separately, formalin tissue biopsies from the same animals were taken post-mortem and embedded in paraffin. The paraffin tissue blocks were sectioned and stained for morphology and presence of collagen deposits.

### Example 14

Comparison to Known Tracer Compounds

A comparison of [⁶⁸Ga]Ga-1 ([⁶⁸Ga]Ga-DOTA-NH—(CH₂CH₂O)₂—CH₂—C(O)-LRELHLNNN—OH) was made with the following tracer compounds reported in the literature: [⁶⁸Ga]Ga—CBP8 (Sci. Trans. Med. 9, 2017, 1-11), [⁶⁸Ga]Ga-NOTA-Collaglin (Nuclear Medicine and Biology, 41 (2014) 728-736) and [⁶⁸Ga]Ga-NODAGA-Collaglin (Nuclear Medicine and Biology, 41 (2014) 728-736).

[⁶⁸Ga]Ga-1 ([⁶⁸Ga]Ga—NH—(CH₂CH₂O)₂—CH₂—C(O)-LRELHLNNN—OH) was prepared as described above in Example 4.

The biodistribution of putative collagen type I binding peptides [⁶⁸Ga]Ga-DOTA-CBP8, [⁶⁸Ga]Ga-NOTA-Collagelin and [⁶⁸Ga]Ga-NODAGA-Collagelin was obtained from published reports (references 1-2).

The biodistribution of [⁶⁸Ga]Ga-1 ([⁶⁸Ga]Ga—NH—(CH₂CH₂O)₂—CH₂—C(O)-LRELHLNNN—OH) was carried out as described above.

Table V below shows the resulting SUV values for the tested compounds 60 minutes post injection in kidney and liver, respectively.

TABLE V

| | | Kid- | | |
|---|---|---|---|---|
| Tracer | PET tracer | ney | Liver | Animal |
| [⁶⁸Ga]Ga-1 | [⁶⁸Ga]Ga-DOTA- | 1.7 | 0.7 | Rat |
| | NH—(CH₂CH₂O)₂—CH₂—C(O)- | | | |
| | LRELHLNNN-OH | | | |
| Comparative | [⁶⁸Ga]Ga-DOTA-CBP8 | 10 | 0.4 | Mouse |
| Tracer 1 | | | | |
| Comparative | [⁶⁸Ga]Ga-NOTA-Collagelin | 6.2 | 0.5 | Rat |
| Tracer 2 | | | | |
| Comparative | [⁶⁸Ga)Ga-NODAGA- | 11.1 | 0.8 | Rat |
| Tracer 3 | Collagelin | | | |

As seen from FIG. 10, [⁶⁸Ga]Ga-DOTA-NH—(CH₂CH₂O)₂—CH₂—C(O)-LRELHLNNN—OH ([⁶⁸Ga]Ga-1) demonstrated rapid clearance from most tissues (SUV<1 after 60 minutes). [⁶⁸Ga]Ga-1 exhibited renal excretion, but importantly also unusually low re-uptake into the renal cortex. Almost all radiolabeled peptide exited the circulation into urine. The kidney background signal was therefore low (SUV≈1) 2h after administration. A comparison of [⁶⁸Ga]Ga-1 and Comparative Tracer 2 in Table V shows that replacement of the cyclic peptide CBP8 with the linear peptide LRELHLNNN lowered the SUV value for kidney from 10 to 1.7 indicating lower non-specific renal retention. Further, the collagelin tracers of Comparative Tracers 3 and 4 had considerably higher SUV values for kidney than the [⁶⁸Ga]Ga-1 tracer. For liver, the [⁶⁸Ga]Ga-1 tracer 1 had a substantially equal value or somewhat higher SUV value than the Comparative Tracers 1-3. It was concluded that the compounds of the present disclosure, such as the compound of the [⁶⁸Ga]Ga-1 tracer in Table V above, are generally useful for tracing fibrosis. In particular, the compounds of the present disclosure were found to be useful for tracing fibrosis in kidney.

### REFERENCES

1. Nuclear Medicine and Biology, 41 (2014) 728-736.
2. Sci. Trans. Med. 9, 2017, 1-11
3. WO 2018/053276

1. A composition comprising:
(i) a compound of Formula I: 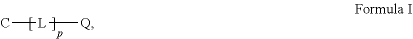
or a pharmaceutically acceptable salt thereof,
and
(ii) a nuclide M, or a pharmaceutically acceptable salt thereof,
wherein
C is a chelator selected from the group consisting of: 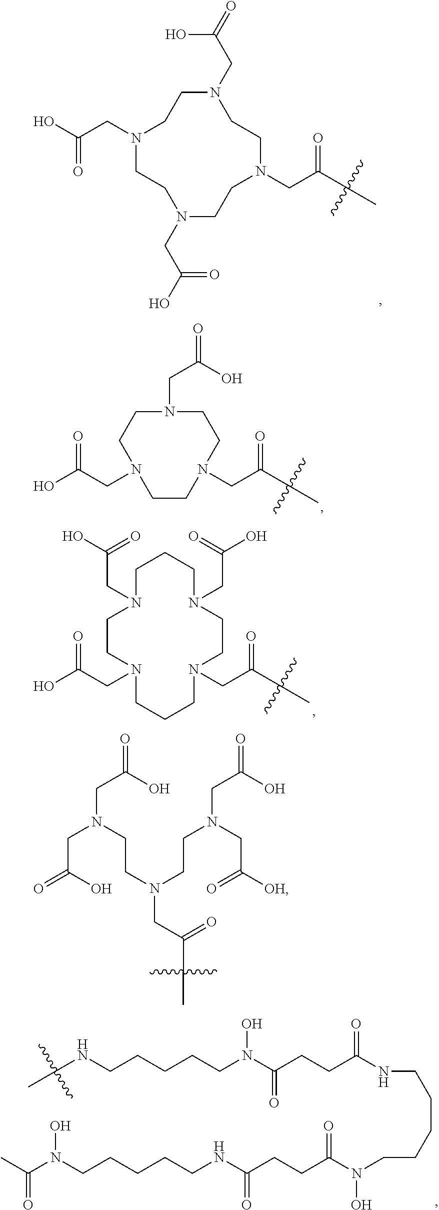 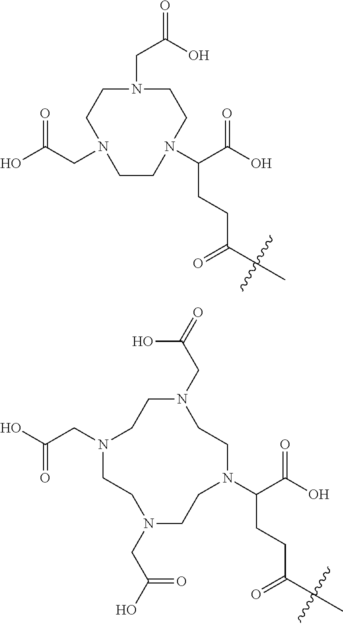 [SMILES: *C(=O)CCC(C(=O)O)N1CCN(CC(=O)O)CCN(CC(=O)O)CC1 ; *C(=O)CCC(C(=O)O)N1CCN(CC(=O)O)CCN(CC(=O)O)CCN(CC(=O)O)CC1]
and a derivative of any one of the foregoing chelators wherein one or more of the carboxylic acid groups of the chelator have been converted into an ester group or an amide group,
L is a linker: 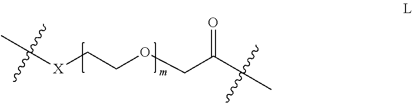
wherein
m is an integer within the range of from 1 to 20, and
X is NH or C(O) and forms an amide bond C(O)NH, with a C(O) or NH moiety of the chelator,
p is 0 or 1,
Q is a peptide of
SEQ ID NO: 1, a peptide analogue of SEQ ID NO: 1 having at least 88.8% identity to SEQ ID NO: 1, and/or
a peptide of SEQ ID NO: 1, a peptide analogue of SEQ ID NO: 1 having at least 88.8% identity to SEQ ID NO: 1 in which the C-terminal COOH is replaced with CONH₂,
and
M is selected from the group consisting of ⁶⁸Ga, ¹⁸F, ⁶⁴Cu, ⁴⁴Sc, ⁸⁹Zr, ¹¹¹In, ⁶⁷Ga, ^{99m}Tc, ¹⁷⁷Lu, ^{86/90}Y, Mn and Gd.

2. The composition according to claim 1, wherein the composition comprises a compound of Formula II: 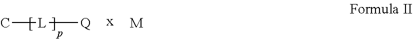
or a pharmaceutically acceptable salt thereof,
said compound of Formula II being a combination of
(i) the compound of Formula I and
(ii) the nuclide M,
wherein (i) and (ii) are provided in a ratio (i)/(ii) equal to one.

3. The composition according to claim 1, wherein the compound of Formula I is selected from the group consisting of a compound of Formula Ia, Formula Ib, Formula Ic, Formula Id, Formula Ie, Formula If or Formula Ig 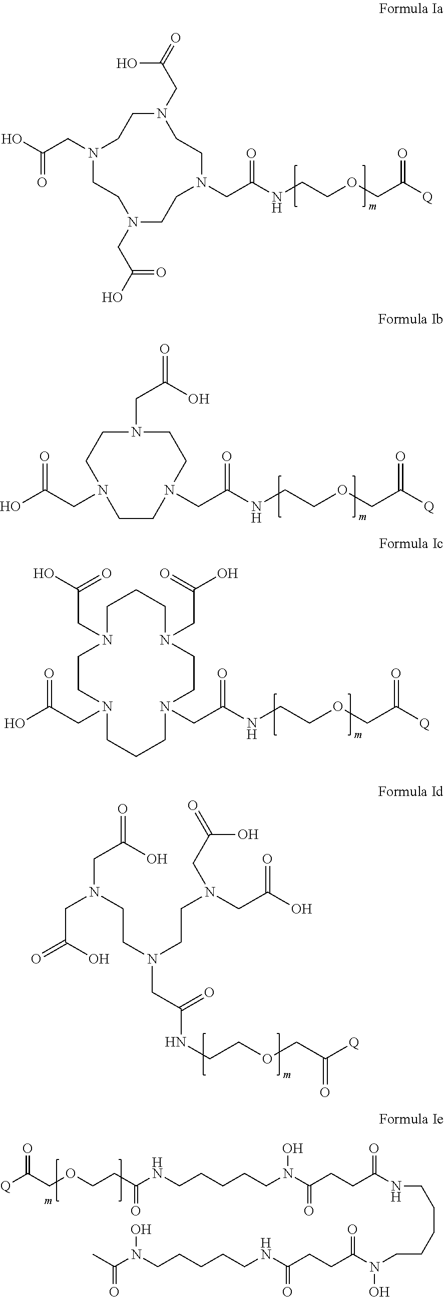 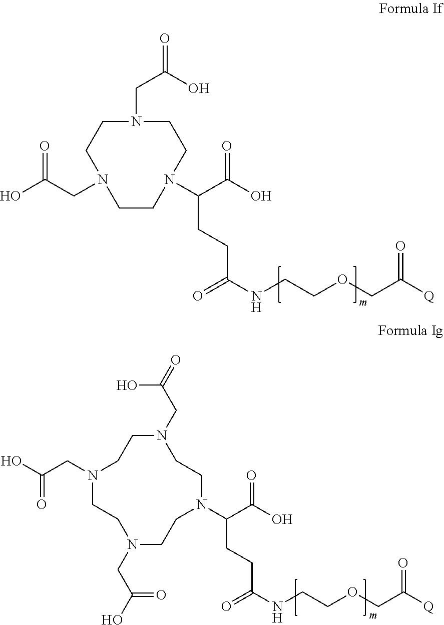
or a derivative of any one of the foregoing compounds wherein one or more of the carboxylic acid groups of the chelator have been converted into an ester group or an amide group,
or a pharmaceutically acceptable salt of any one of the foregoing compounds or of a derivative of any one of the foregoing compounds.

4. The composition according to claim 1, wherein the compound of Formula I is selected from the group consisting of
DOTA-NH—(CH₂CH₂O)₂—CH₂—C(O)-L-R-E-L-H-L-N—N—N—OH, Compound 15
DOTA-NH—(CH₂CH₂O)₂—CH₂—C(O)-L-R-E-L-H-L-N-A-N—OH, Cmpd 2
DOTA-NH—(CH₂CH₂O)₂—CH₂—C(O)-L-R-E-L-H-L-N—N—N—NH₂, Cmpd 3
DOTA-NH—(CH₂CH₂O)₂—CH₂—C(O)-L-R-E-L-H-L-N—N-n-OH, Cmpd 4
DOTA-NH—(CH₂CH₂O)₂—CH₂—C(O)-L-R-E-L-H-L-N-A-N—NH₂, Cmpd 5
DOTA-NH—(CH₂CH₂O)₂—CH₂—C(O)-L-R-E-L-H—V-N—N—N—OH, Cmpd 6
DOTA-NH—(CH₂CH₂O)₂—CH₂—C(O)-L-R-E-1-H-L-N—N—N—OH, Cmpd 7
DOTA-NH—(CH₂CH₂O)₂—CH₂—C(O)-L-R-E-L-H-L-N—N—OH, Cmpd 8
DOTA-NH—(CH₂CH₂O)₂—CH₂—C(O)—H-L-R-E-L-H-L-N—N—N—OH, Cmpd 9
DOTA-NH—(CH₂CH₂O)₂—CH₂—C(O)-L-R-E-L-H-L-N—N—N—K—OH, Cmpd 10
DOTA-NH—(CH₂CH₂O)₃—CH₂—C(O)-L-R-E-L-H-L-N—N—N—OH, Cmpd 11
DOTA-L-R-E-L-H-L-N—N—N—OH, Cmpd 12
H₂N-L-R-E-L-H-L-N—N—N—K(DOTA-NH—(CH₂CH₂O)₂—CH₂—C(O))—NH₂, Cmpd 13
H₂N-L-R-E-K(DOTA-NH—(CH₂CH₂O)₂—CH₂—C(O))—H-L-N—N—N—OH, Cmpd 14
H₂N-L-R-E-L-H—K(DOTA-NH—(CH₂CH₂O)₂—CH₂—C(O))—N—N—N—OH, and Cmpd 15
NOTA-NH—(CH₂CH₂O)₂—CH₂—C(O)-L-R-E-L-H-L-N—N—N—OH Cmpd 16.

5. The composition according to claim 4, wherein the compound of Formula I is selected from the croup consisting of
DOTA-NH—(CH₂CH₂O)₂—CH₂—C(O)-L-R-E-L-H-L-N—N—N—OH, Cmpd 1
DOTA-NH—(CH₂CH₂O)₂—CH₂—C(O)-L-R-E-L-H-L-N—N—N—NH₂, Cmpd 3
DOTA-NH—(CH₂CH₂O)₂—CH₂—C(O)-L-R-E-L-H-L-N—N-n-OH, Cmpd 4
DOTA-NH—(CH₂CH₂O)₂—CH₂—C(O)-L-R-E-L-H-L-N-A-N—NH₂, Cmpd 5
DOTA-NH—(CH₂CH₂O)₂—CH₂—C(O)-L-R-E-L-H—V-N—N—N—OH, Cmpd 6
DOTA-NH—(CH₂CH₂O)₃—CH₂—C(O)-L-R-E-L-H-L-N—N—N—OH, Cmpd 11
DOTA-L-R-E-L-H-L-N—N—N—OH, Cmpd 12
H₂N-L-R-E-L-H-L-N—N—N—K(DOTA-NH—(CH₂CH₂O)₂—CH₂—C(O))—NH₂, and Cmpd 13
NOTA-NH—(CH₂CH₂O)₂—CH₂—C(O)-L-R-E-L-H-L-N—N—N—OH Cmpd 16.

6. The composition according to claim 4, wherein the compound of Formula I is selected from the croup consisting of
DOTA-NH—(CH₂CH₂O)₂—CH₂—C(O)-L-R-E-L-H-L-N—N—N—OH, Cmpd 1
DOTA-NH—(CH₂CH₂O)₂—CH₂—C(O)-L-R-E-L-H-L-N-A-N—OH, Cmpd 2
DOTA-NH—(CH₂CH₂O)₂—CH₂—C(O)-L-R-E-L-H-L-N—N—N—NH₂, Cmpd 3
DOTA-NH—(CH₂CH₂O)₂—CH₂—C(O)-L-R-E-L-H-L-N-A-N—NH₂, and Cmpd 5
NOTA-NH—(CH₂CH₂O)₂—CH₂—C(O)-L-R-E-L-H-L-N—N—N—OH Cmpd 16.

7. The composition according to claim 4, wherein the compound of Formula I is
DOTA-NH—(CH₂CH₂O)₂—CH₂—C(O)-L-R-E-L-H-L-N-A-N—NH₂ Cmpd 5.

8. The composition according to claim 1, wherein M is
(i)⁶⁸Ga, ¹⁸F, ⁶⁴Cu, ¹¹¹In, ^{99m}Tc, Gd, ¹⁷⁷Lu and ^{86/90}Y
(ii)⁶⁸Ga, or
(iii)¹⁸F.

9. The composition according to claim 1, wherein said composition is a pharmaceutical composition further comprising a pharmaceutically acceptable carrier, excipient and/or diluent.

10. The composition according to claim 1, for use in diagnosing and/or monitoring of fibrosis.

11. The composition according to claim 1, for use in diagnosing and/or monitoring the extent of fibrosis in a patient suffering from, suspected to be suffering from and/or being treated for, fibrosis.

12. The composition for use according to claim 10, wherein the fibrosis is one or more of the following: liver fibrosis, kidney fibrosis, heart fibrosis, pancreas fibrosis, brain fibrosis, lung fibrosis such as idiopathic pulmonary fibrosis.

13. The composition for use according to claim 10, wherein the diagnosing and/or monitoring of fibrosis involves diagnosing and/or monitoring of the extent of fibrosis.

14. The composition for use according to claim 10, wherein the diagnosing and/or monitoring involves imaging, such as Positron Emission Tomography (PET), Single-Photon Emission Computed Tomography (SPECT) or Magnetic Resonance Imaging (MRI), taking place
ex vivo, and/or
in vivo such as in a patient.

15-17. (canceled)

18. A compound of Formula I as defined in claim 7 or a pharmaceutically acceptable salt thereof.

19. A compound of Formula II as defined in claim 2 or a pharmaceutically acceptable salt thereof.

20. The compound according to claim 19, for use as an imaging agent.

21. The compound according to claim 19, for use in diagnosing and/or monitoring of fibrosis.

22. The compound according to claim 21, for use in diagnosing and/or monitoring the extent of fibrosis in a patient suffering from, suspected to be suffering from and/or being treated for, fibrosis.

23. The compound for use according to claim 22, wherein the fibrosis is one or more of the following: liver fibrosis, kidney fibrosis, heart fibrosis, pancreas fibrosis, brain fibrosis, lung fibrosis such as idiopathic pulmonary fibrosis.

24. The compound for use according to claim 21, wherein the diagnosing and/or monitoring involves imaging, such as Positron Emission Tomography (PET), Single-Photon Emission Computed Tomography (SPECT) or Magnetic Resonance Imaging (MRI), taking place
ex vivo, and/or
in vivo such as in a patient.

25. A method for the diagnosis and/or monitoring of fibrosis, said method comprising the steps of:
a) administering an imaging agent from one or more of the following:
the composition according to claim 1,
to a patient suffering from, suspected to be suffering from and/or being treated for, fibrosis;
b) subjecting the patient to a medical imaging technique, such as Positron Emission Tomography (PET), Single-Photon Emission Computed Tomography (SPECT) or Magnetic Resonance Imaging (MRI) imaging, and recording signals from the imaging agent administered in step a),
c) determining and/or monitoring if the patient suffers from fibrosis, and
d) optionally determining the extent of the fibrosis.

26-27. (canceled)
